(19) United States
(12) Patent Application Publication
(10) Pub. No.: US 2022/0081407 A1
(43) Pub. Date: Mar. 17, 2022

(54) METHODS OF PREPARING SUBSTITUTED INDANES

(71) Applicant: Peloton Therapeutics, Inc., Kenilworth, NJ (US)

(72) Inventors: Peter J. Stengel, Longmont, CO (US); Rui Xu, Dallas, TX (US); Stacey Renee Spencer, Lyons, CO (US)

(73) Assignee: Peloton Therapeutics, Inc., Kenilworth, NJ (US)

(21) Appl. No.: 17/017,864

(22) Filed: Sep. 11, 2020

(51) Int. Cl. C07D 317/72; C07C 319/20; C07C 315/04

(52) U.S. Cl. CPC C07D 317/72; C07C 321/28; C07C 315/04; C07C 319/20

(57) ABSTRACT

This application discloses methods for the synthesis of substituted indane analogs that modulate HIF-2α activity, as well as key intermediates produced thereby. The synthesis of 3-(((1S,2S,3R)-2,3-difluoro-1-hydroxy-7-(methylsulfonyl)-2,3-dihydro-1H-inden-4-yl)oxy)-5-fluorobenzonitrile was exemplified.

### CROSS REFERENCE TO RELATED APPLICATIONS

This application is a U.S. non-provisional application which claims the benefit of provisional Application No. 62/901,669, filed Sep. 17, 2019.

### BACKGROUND OF THE INVENTION

An adequate supply of oxygen to tissues is essential in maintaining mammalian cell function and physiology. A deficiency in oxygen supply to tissues is a characteristic of a number of pathophysiologic conditions in which there is insufficient blood flow to provide adequate oxygenation. The hypoxic (low oxygen) environment of tissues activates a signaling cascade that drives the induction or repression of the transcription of a multitude of genes implicated in events such as angiogenesis (neo-vascularization), glucose metabolism, and cell survival/death. A key to this hypoxic transcriptional response lies in the transcription factors, the hypoxia-inducible factors (HIF). HIFs are dysregulated in a vast array of cancers through hypoxia-dependent and independent mechanisms and expression is associated with poor patient prognosis.

HIFs consist of an oxygen-sensitive HIFα subunit and a constitutively expressed HIFβ subunit. When HIFs are activated, the HIFα and HIFβ subunits assemble a functional heterodimer (the a subunit heterodimerizes with the β subunit). Both HIFα and HIFβ have two identical structural characteristics, a basic helix-loop-helix (bHLH) and PAS domains (PAS is an acronym referring to the first proteins, PER, ARNT, SIM, in which this motif was identified). There are three human HIFα subunits (HIF-1α, HIF-2α, and HIF-3α) that are oxygen sensitive. Among the three subunits, HIF-1α is the most ubiquitously expressed and induced by low oxygen concentrations in many cell and tissue types. HIF-2α is highly similar to HIF-la in both structure and function, but exhibits more restricted cell and tissue-specific expression, and might also be differentially regulated by nuclear translocation. HIF-3α also exhibits conservation with HIF-1α and HIF-2α in the HLH and PAS domains. HIF-1β (also referred to as ARNT—Aryl Hydrocarbon Receptor Nuclear Translocator), the dimerization partner of the HIFα subunits, is constitutively expressed in all cell types and is not regulated by oxygen concentration.

### SUMMARY OF THE INVENTION

In certain aspects, the present disclosure provides a method for preparing a compound of Formula (I):

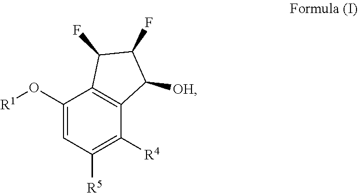

comprising:

(i) contacting a compound of Formula (II):

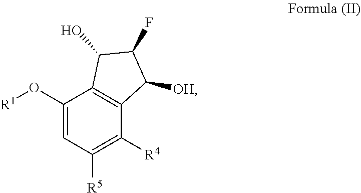

with a fluorinating reagent to generate a compound of Formula (I), wherein:

R¹ is aryl or heteroaryl;

R⁴ is sulfinyl, sulfonamide, sulfonyl or sulfoximinyl; and

R⁵ is hydrogen, halo or alkyl.

In certain embodiments, the fluorinating agent is selected from bis(2-methoxyethyl)aminosulfur trifluoride (Deoxo-Fluor®), perfluoro-1-butanesulfonyl fluoride (PB SF), 2-pyridinesulfonyl fluoride (PyFluor), 1,3-bis(2,6-diisopropylphenyl)-2,2-difluoro-4-imidazoline (PhenoFluor), 1,3-bis(2,6-diisopropylphenyl)-2-fluoroimidazolium tetrafluoroborate (AlkylFluor), sulfur tetrafluoride, diethylaminosulfur trifluoride (DAST), and morpholinosulfur trifluoride. In certain embodiments, the fluorinating agent is perfluoro-1-butanesulfonyl fluoride. In certain embodiments, the fluorinating agent is bis(2-methoxyethyl)aminosulfur trifluoride (Deoxo-Fluor®).

In certain embodiments, the reaction further comprises a base. In certain embodiments, the base is 1,8-diazabicyclo[5.4.0]undec-7-ene (DBU).

In certain embodiments, the reaction further comprises an aprotic solvent. In certain embodiments, the aprotic solvent comprises tetrahydrofuran, 2-methyltetrahydrofuran, ethyl acetate, dioxane, 1,2-dimethyoxyethane, or a combination thereof. In certain embodiments, the aprotic solvent is 1,2-dimethoxyethane.

In certain embodiments, the fluorinating agent is perfluoro-1-butanesulfonyl fluoride and the reaction further comprises DBU and 1,2-dimethoxyethane.

In certain embodiments, the reaction is maintained at a temperature between about −80° C. and about 30° C. In certain embodiments, the reaction is maintained at a temperature between about 15° C. and about 20° C. In certain embodiments, the reaction is maintained at a temperature between about 2° C. and about 8° C.

In certain embodiments, the method further comprises recrystallizing the compound of Formula (I) in a suitable solvent. In certain embodiments, the suitable solvent is a mixture of acetonitrile and water.

In certain embodiments, the method further comprises, prior to step (i):

(i-b) contacting a compound of Formula (IV):

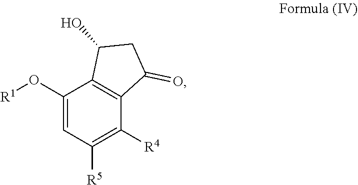

with a fluorinating reaction to form an intermediate compound of Formula (III):

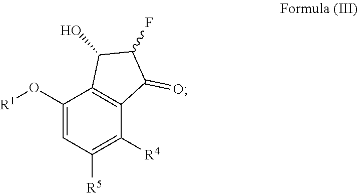

and

(i-a) reducing the compound of Formula (III) to generate the compound of Formula (II).

In another aspect, the present disclosure provides a method for preparing a compound of Formula (II):

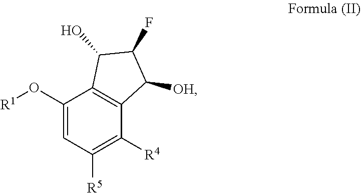

comprising:

(i-b) contacting a compound of Formula (IV):

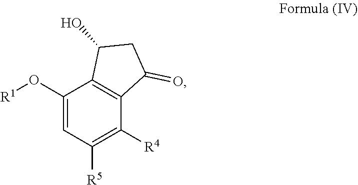

with a fluorinating reaction to form an intermediate compound of Formula (III):

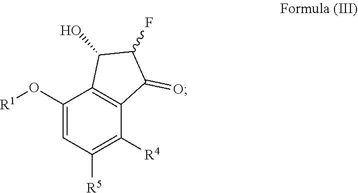

and

(i-a) reducing the compound of Formula (III) to generate the compound of Formula (II), wherein:

R¹ is aryl or heteroaryl;

R⁴ is sulfinyl, sulfonamide, sulfonyl or sulfoximinyl; and

R⁵ is hydrogen, halo or alkyl.

In certain embodiments, the fluorinating reagent of (i-b) is 1-chloromethyl-4-fluoro-1,4-diazoniabicyclo[2.2.2]octane bis(tetrafluoroborate).

In certain embodiments, step (i-b) further comprises an acid. In certain embodiments, the acid is sulfuric acid.

In certain embodiments, step (i-b) further comprises a solvent. In certain embodiments, the solvent is selected from methanol, acetonitrile, or a combination thereof.

In certain embodiments, step (i-b) is maintained at a temperature between about 50° C. and about 70° C.

In certain embodiments, the reducing of step (i-a) is an asymmetric reduction. In certain embodiments, the reduction is a Noyori reduction.

In certain embodiments, step (i-a) comprises a chiral ruthenium catalyst. In certain embodiments, the ruthenium catalyst is ((R,R)-2-amino-1,2-diphenylethyl)-[(4-tolyl)sulfonyl]-amido(p-cymene)-ruthenium(II) chloride.

In certain embodiments, step (i-a) comprises a hydrogen or a hydride source.

In certain embodiments, step (i-a) comprises formic acid.

In certain embodiments, step (i-a) comprises a base. In certain embodiments, the base is triethylamine.

In certain embodiments, step (i-a) comprises a solvent. In certain embodiments, the solvent is ethyl acetate.

In certain embodiments, step (i-a) is maintained at a temperature between about 10° C. and about 35° C.

In certain embodiments, the method further comprises, prior to step (i-b):

(i-d) subjecting a compound of Formula (VI):

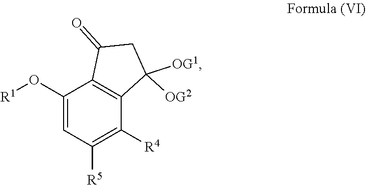

to a reduction to form an intermediate compound of Formula (V):

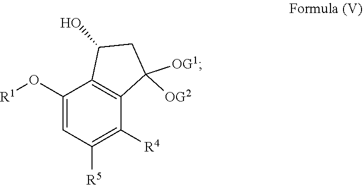

and

(i-c) deprotecting the compound of Formula (V) to generate the compound of Formula (IV), wherein:

G¹ and G² are independently selected from alkyl, alkenyl and aryl, or G¹ and G², together with the carbon atom to which they are attached, form a 5- or 6-membered heterocycle.

In another aspect, the present disclosure provides a method for preparing a compound of Formula (IV):

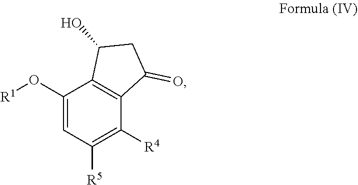

comprising:

(i-d) subjecting a compound of Formula (VI):

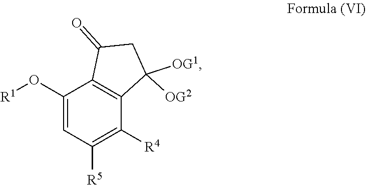

to a reduction to form an intermediate compound of Formula (V):

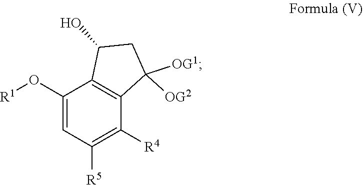

and

(i-c) deprotecting the compound of Formula (V) to generate the compound of Formula (IV), wherein:

R¹ is aryl or heteroaryl;

R⁴ is sulfinyl, sulfonamide, sulfonyl or sulfoximinyl;

R⁵ is hydrogen, halo or alkyl; and

G¹ and G² are independently selected from alkyl, alkenyl and aryl, or G¹ and G², together with the carbon atom to which they are attached, form a 5- or 6-membered heterocycle.

In certain embodiments, the reduction of step (i-d) is an asymmetric reduction. In certain embodiments, the reduction is a Noyori reduction.

In certain embodiments, step (i-d) comprises a chiral ruthenium catalyst. In certain embodiments, the ruthenium catalyst is ((R,R)-2-amino-1,2-diphenylethyl)-[(4-tolyl)sulfonyl]-amido(p-cymene)-ruthenium(II) chloride.

In certain embodiments, step (i-d) comprises a hydrogen or a hydride source.

In certain embodiments, step (i-d) comprises formic acid.

In certain embodiments, step (i-d) comprises a base. In certain embodiments, the base is triethylamine.

In certain embodiments, step (i-d) comprises a solvent. In certain embodiments, the solvent is ethyl acetate.

In certain embodiments, step (i-d) is maintained at a temperature between about 10° C. and about 35° C.

In certain embodiments, step (i-c) comprises an acid. In certain embodiments, the acid is hydrochloric acid.

In certain embodiments, step (i-c) comprises a solvent. In certain embodiments, the solvent is acetone, 2-butanone, or a combination thereof.

In certain embodiments, step (i-c) is maintained at a temperature between about 10° C. and about 35° C.

In certain embodiments, R¹ is phenyl or pyridyl. In certain embodiments, R¹ is phenyl. In certain embodiments, R¹ is substituted with one or more substituents selected from halo, C₁₋₄ alkyl, C₁₋₄ alkoxy and cyano. In certain embodiments, R¹ is substituted with fluoro and cyano.

In certain embodiments, R⁴ is selected from —SO₂CH₃, —SO₂NH₂, —SO₂CF₃, —S(═O)(N—CN)CH₂CH₂F, and —S(═O)(N—CN)CH₃. In certain embodiments, R⁴ is —SO₂CH₃.

In certain embodiments, R⁵ is hydrogen.

In certain embodiments, R¹ is phenyl, wherein said phenyl is substituted with fluoro and cyano; R⁴ is —SO₂CH₃; and R⁵ is hydrogen.

In certain embodiments, the compound of Formula (I) is represented by Formula (I-A):

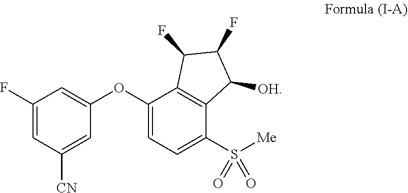 [SMILES: CS(=O)(=O)c1ccc(Oc2cc(F)cc(C#N)c2)c2c1[C@H](O)[C@H](F)[C@@H]2F]

In certain embodiments, the compound of Formula (II) is represented by Formula (II-A):

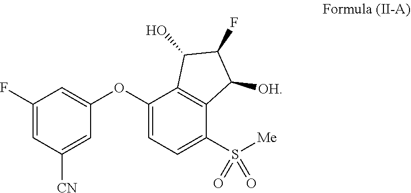 [SMILES: CS(=O)(=O)c1ccc(Oc2cc(F)cc(C#N)c2)c2c1[C@H](O)[C@H](F)[C@H]2O]

In certain embodiments, the compound of Formula (III) is represented by Formula (III-A):

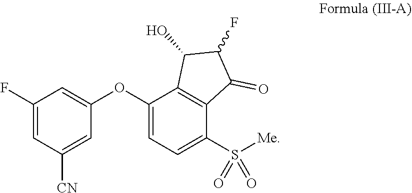 [SMILES: CS(=O)(=O)c1ccc(Oc2cc(F)cc(C#N)c2)c2c1C(=O)C(F)[C@H]2O]

In certain embodiments, the compound of Formula (IV) is represented by Formula (IV-A):

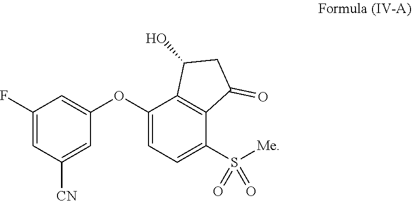 [SMILES: CS(=O)(=O)c1ccc(Oc2cc(F)cc(C#N)c2)c2c1C(=O)C[C@H]2O]

In certain embodiments, the compound of Formula (V) is represented by Formula (V-A):

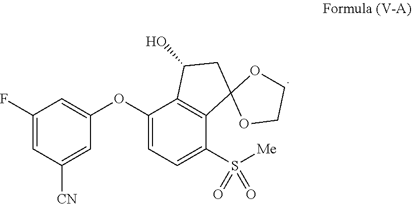 [SMILES: CS(=O)(=O)c1ccc(Oc2cc(F)cc(C#N)c2)c2c1C1(C[C@H]2O)OCCO1]

In certain embodiments, the compound of Formula (VI) is represented by Formula (VI-A):

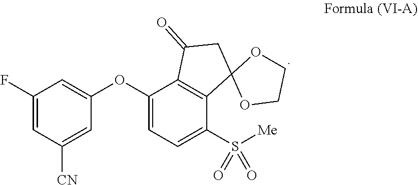 [SMILES: CS(=O)(=O)c1ccc(Oc2cc(F)cc(C#N)c2)c2c1C1(CC2=O)OCCO1]

In another aspect, the present disclosure provides compound of Formula (I-A):

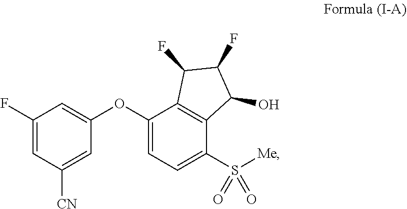 [SMILES: CS(=O)(=O)c1ccc(Oc2cc(F)cc(C#N)c2)c2c1[C@H](O)[C@H](F)[C@@H]2F]

obtained by the method of any one of the preceding claims.

In another aspect, the present disclosure provides a composition comprising, by weight relative to the total weight of the composition, at least 97% of a compound of Formula (I-A). In certain embodiments, the compound is characterized by an enantiomeric excess of at least 98%. In certain embodiments, the composition comprises, by weight relative to the total weight of the composition, less than 0.5% water. In certain embodiments, the composition comprises less than 100 ppm ruthenium.

In another aspect, the present disclosure provides a compound of Formula (IV)

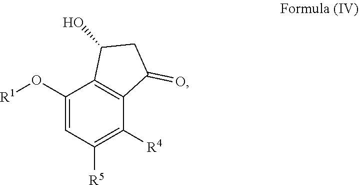

wherein:

R¹ is aryl or heteroaryl;

R⁴ is sulfinyl, sulfonamide, sulfonyl or sulfoximinyl; and

R⁵ is hydrogen, halo or alkyl.

In certain embodiments, the compound of Formula (IV) is represented by Formula (IV-A):

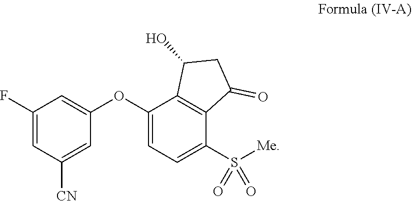 [SMILES: CS(=O)(=O)c1ccc(Oc2cc(F)cc(C#N)c2)c2c1C(=O)C[C@H]2O]

In another aspect, the present disclosure provides a compound of Formula (III):

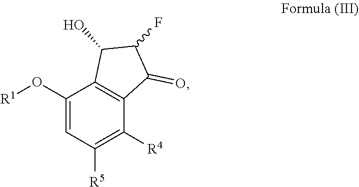

wherein:

R¹ is aryl or heteroaryl;

R⁴ is sulfinyl, sulfonamide, sulfonyl or sulfoximinyl; and

R⁵ is hydrogen, halo or alkyl.

In certain embodiments, the compound of Formula (III) is represented by Formula (III-A):

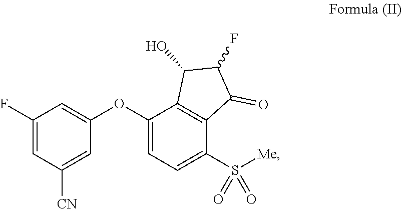 [SMILES: CS(=O)(=O)c1ccc(Oc2cc(F)cc(C#N)c2)c2c1C(=O)C(F)[C@H]2O]

a fluorinating reagent, a base and a solvent, wherein:

R¹ is aryl or heteroaryl;

R⁴ is sulfinyl, sulfonamide, sulfonyl or sulfoximinyl; and

R⁵ is hydrogen, halo or alkyl.

In another aspect, the present disclosure provides a reaction mixture comprising a compound of Formula (III):

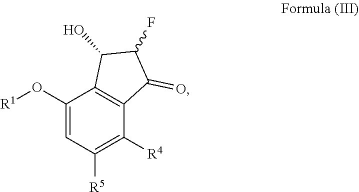

a ruthenium catalyst, a hydride source and a solvent, wherein:

R¹ is aryl or heteroaryl;

R⁴ is sulfinyl, sulfonamide, sulfonyl or sulfoximinyl; and

R⁵ is hydrogen, halo or alkyl.

In another aspect, the present disclosure provides a reaction mixture comprising a compound of Formula (IV):

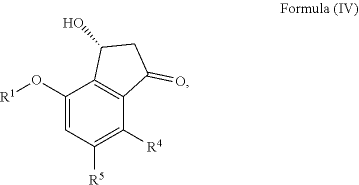

a fluorinating reagent, an acid and a solvent, wherein:

R¹ is aryl or heteroaryl;

R⁴ is sulfinyl, sulfonamide, sulfonyl or sulfoximinyl; and

R⁵ is hydrogen, halo or alkyl.

In another aspect, the present disclosure provides a reaction mixture comprising a compound of Formula (V):

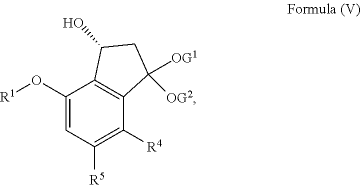

an acid and a solvent, wherein:

R¹ is aryl or heteroaryl;

R⁴ is sulfinyl, sulfonamide, sulfonyl or sulfoximinyl;

R⁵ is hydrogen, halo or alkyl; and

G¹ and G² are independently selected from alkyl, alkenyl and aryl, or G¹ and G², together with the carbon atom to which they are attached, form a 5- or 6-membered heterocycle.

In another aspect, the present disclosure provides a reaction mixture comprising a compound of Formula (VI):

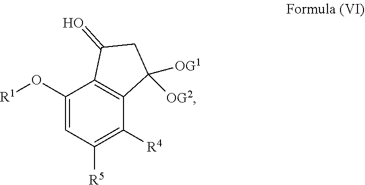

a ruthenium catalyst, a hydride source and a solvent, wherein:

R¹ is aryl or heteroaryl;

R⁴ is sulfinyl, sulfonamide, sulfonyl or sulfoximinyl;

R⁵ is hydrogen, halo or alkyl; and

G¹ and G² are independently selected from alkyl, alkenyl and aryl, or G¹ and G², together with the carbon atom to which they are attached, form a 5- or 6-membered heterocycle.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

FIG. 1 shows a ¹H NMR spectrum for 3-(((1S,2S,3R)-2,3-difluoro-1-hydroxy-7-(methylsulfonyl)-2,3-dihydro-1H-inden-4-yl)oxy)-5-fluorobenzonitrile.

FIG. 2 shows an FTIR spectrum for 3-(((1S,2S,3R)-2,3-difluoro-1-hydroxy-7-(methylsulfonyl)-2,3-dihydro-1H-inden-4-yl)oxy)-5-fluorobenzonitrile.

FIG. 3 shows an XRPD pattern of crystalline 3-(((1S,2S,3R)-2,3-difluoro-1-hydroxy-7-(methylsulfonyl)-2,3-dihydro-1H-inden-4-yl)oxy)-5-fluorobenzonitrile.

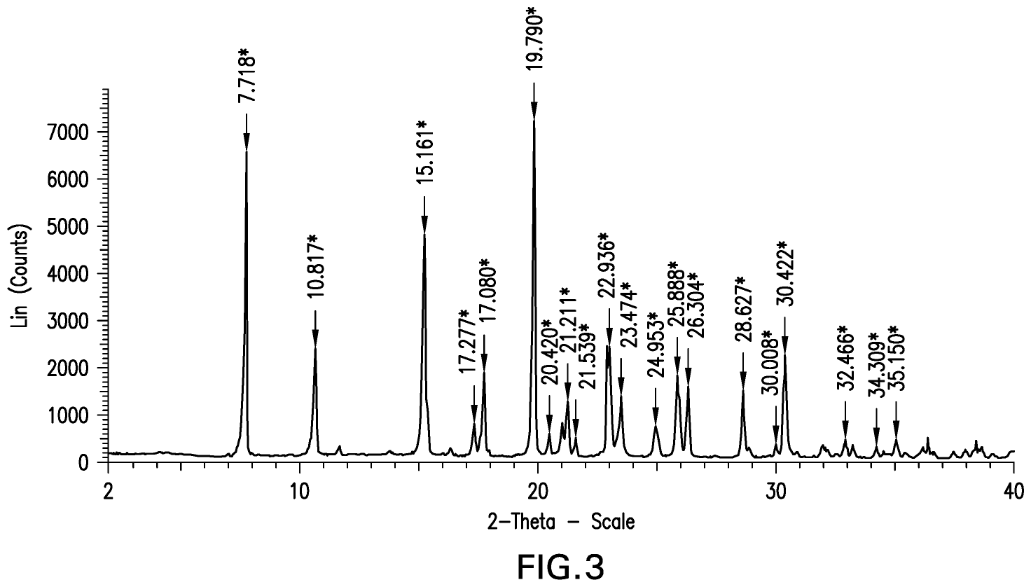

### DETAILED DESCRIPTION OF THE INVENTION

Good manufacturing practices are usually required for large scale manufacture of clinically useful drug candidates. Compounds of Formula (I), and the compound of Formula (I-A) in particular, are potent HIF-2α inhibitors. Compounds of Formulae (I) and (I-A) in particular have been tested in a HIF-2α scintillation proximity assay (Example 348, WO2015035223), demonstrating potent activity in disrupting the binding between a radio-labeled ligand and HIF-2α PAS-B domain. The compound of Formula (I-A), also known as PT2977, has shown encouraging outcomes in patients with advanced renal cell carcinoma. Data support continued clinical development of PT2977 as a monotherapy or in combination with other agents.

The present invention further includes the compounds of Formulae (I) (I-A) and in all their isolated forms. For example, the above-identified compounds are intended to encompass all forms of the compounds such as, any solvates, co-crystals, hydrates, stereoisomers, and tautomers thereof.

Provided herein are certain processes and methods for preparing a compound of Formula (I) and the compound of Formula (I-A) in particular:

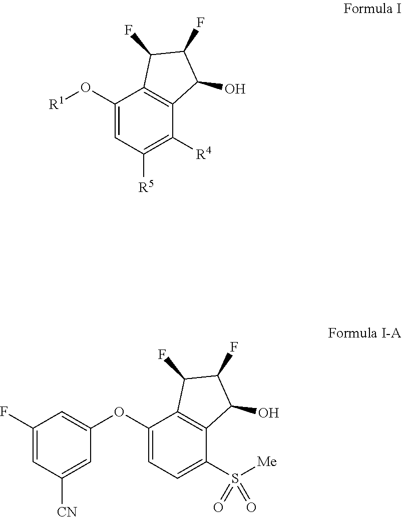

wherein:

R¹ is aryl or heteroaryl;

R⁴ is sulfinyl, sulfonamide, sulfonyl or sulfoximinyl; and

R⁵ is hydrogen, halo or alkyl.

The processes and methods provided herein overcome certain manufacturing drawbacks and allow for the synthesis of high purity compounds while reducing waste and/or by-products. The methods described herein allow for large-scale production compliant with current good manufacturing practice (GMP) guidelines.

Unless defined otherwise, all technical and scientific terms used herein have the same meaning as is commonly understood by one of skill in the art to which this invention belongs.

As used in the specification and claims, the singular form “a”, “an” and “the” include plural references unless the context clearly dictates otherwise.

The term “about” as used herein refers to ±10% of a stated value.

The term “C_{x-y}” or “Cₓ-C_{y}” when used in conjunction with a chemical moiety, such as alkyl, alkenyl, or alkynyl is meant to include groups that contain from x to y carbons in the chain. For example, the term “C_{x-y} alkyl” refers to substituted or unsubstituted saturated hydrocarbon groups, including straight-chain alkyl and branched-chain alkyl groups that contain from x to y carbons in the chain.

“Alkyl” refers to substituted or unsubstituted saturated hydrocarbon groups, including straight-chain alkyl and branched-chain alkyl groups. An alkyl group may contain from one to twelve carbon atoms (e.g., C₁₋₁₂ alkyl), such as one to eight carbon atoms (C₁₋₈ alkyl) or one to six carbon atoms (C₁₋₆ alkyl). Exemplary alkyl groups include methyl, ethyl, n-propyl, isopropyl, n-butyl, isobutyl, sec-butyl, tert-butyl, pentyl, isopentyl, neopentyl, hexyl, septyl, octyl, nonyl, and decyl. An alkyl group is attached to the rest of the molecule by a single bond. Unless stated otherwise specifically in the specification, an alkyl group is optionally substituted by one or more substituents such as those substituents described herein.

“Haloalkyl” refers to an alkyl group that is substituted by one or more halogens. Exemplary haloalkyl groups include trifluoromethyl, difluoromethyl, trichloromethyl, 2,2,2-trifluoroethyl, 1,2-difluoroethyl, 3-bromo-2-fluoropropyl, and 1,2-dibromoethyl.

“Alkenyl” refers to substituted or unsubstituted hydrocarbon groups, including straight-chain or branched-chain alkenyl groups containing at least one double bond. An alkenyl group may contain from two to twelve carbon atoms (e.g., C₂-12 alkenyl). Exemplary alkenyl groups include ethenyl (i.e., vinyl), prop-1-enyl, but-1-enyl, pent-1-enyl, penta-1,4-dienyl, and the like. Unless stated otherwise specifically in the specification, an alkenyl group is optionally substituted by one or more substituents such as those substituents described herein.

“Alkynyl” refers to substituted or unsubstituted hydrocarbon groups, including straight-chain or branched-chain alkynyl groups containing at least one triple bond. An alkynyl group may contain from two to twelve carbon atoms (e.g., C₂-12 alkynyl). Exemplary alkynyl groups include ethynyl, propynyl, butynyl, pentynyl, hexynyl, and the like. Unless stated otherwise specifically in the specification, an alkynyl group is optionally substituted by one or more substituents such as those substituents described herein.

“Alkylene” or “alkylene chain” refers to substituted or unsubstituted divalent saturated hydrocarbon groups, including straight-chain alkylene and branched-chain alkylene groups that contain from one to twelve carbon atoms. Exemplary alkylene groups include methylene, ethylene, propylene, and n-butylene. Similarly, “alkenylene” and “alkynylene” refer to alkylene groups, as defined above, which comprise one or more carbon-carbon double or triple bonds, respectively. The points of attachment of the alkylene, alkenylene or alkynylene chain to the rest of the molecule can be through one carbon or any two carbons within the chain. Unless stated otherwise specifically in the specification, an alkylene, alkenylene, or alkynylene group is optionally substituted by one or more substituents such as those substituents described herein.

“Heteroalkyl”, “heteroalkenyl” and “heteroalkynyl” refer to substituted or unsubstituted alkyl, alkenyl and alkynyl groups which respectively have one or more skeletal chain atoms selected from an atom other than carbon, e.g., O, N, P, Si, S or combinations thereof, and wherein the nitrogen, phosphorus, and sulfur atoms may optionally be oxidized and the nitrogen heteroatom may optionally be quaternized. If given, a numerical range refers to the chain length in total. For example, a 3- to 8-membered heteroalkyl has a chain length of 3 to 8 atoms. Connection to the rest of the molecule may be through either a heteroatom or a carbon in the heteroalkyl, heteroalkenyl or heteroalkynyl chain. Unless stated otherwise specifically in the specification, a heteroalkyl, heteroalkenyl, or heteroalkynyl group is optionally substituted by one or more substituents such as those substituents described herein.

“Heteroalkylene”, “heteroalkenylene” and “heteroalkynylene” refer to substituted or unsubstituted alkylene, alkenylene and alkynylene groups which respectively have one or more skeletal chain atoms selected from an atom other than carbon, e.g., O, N, P, Si, S or combinations thereof, and wherein the nitrogen, phosphorus, and sulfur atoms may optionally be oxidized and the nitrogen heteroatom may optionally be quaternized. The points of attachment of the heteroalkylene, heteroalkenylene or heteroalkynylene chain to the rest of the molecule can be through either one heteroatom or one carbon, or any two heteroatoms, any two carbons, or any one heteroatom and any one carbon in the heteroalkyl, heteroalkenyl or heteroalkynyl chain. Unless stated otherwise specifically in the specification, a heteroalkylene, heteroalkenylene, or heteroalkynylene group is optionally substituted by one or more substituents such as those substituents described herein.

“Carbocycle” refers to a saturated, unsaturated or aromatic ring in which each atom of the ring is a carbon atom. Carbocycle may include 3- to 10-membered monocyclic rings, 6- to 12-membered bicyclic rings, and 6- to 12-membered bridged rings. Each ring of a bicyclic carbocycle may be selected from saturated, unsaturated, and aromatic rings. In some embodiments, the carbocycle is an aryl. In some embodiments, the carbocycle is a cycloalkyl. In some embodiments, the carbocycle is a cycloalkenyl. In an exemplary embodiment, an aromatic ring, e.g., phenyl, may be fused to a saturated or unsaturated ring, e.g., cyclohexane, cyclopentane, or cyclohexene. Any combination of saturated, unsaturated and aromatic bicyclic rings, as valence permits, are included in the definition of carbocyclic. Exemplary carbocycles include cyclopentyl, cyclohexyl, cyclohexenyl, adamantyl, phenyl, indanyl, and naphthyl. Unless stated otherwise specifically in the specification, a carbocycle is optionally substituted by one or more substituents such as those substituents described herein.

“Heterocycle” refers to a saturated, unsaturated or aromatic ring comprising one or more heteroatoms. Exemplary heteroatoms include N, O, Si, P, B, and S atoms. Heterocycles include 3- to 10-membered monocyclic rings, 6- to 12-membered bicyclic rings, and 6- to 12-membered bridged rings. Each ring of a bicyclic heterocycle may be selected from saturated, unsaturated, and aromatic rings. The heterocycle may be attached to the rest of the molecule through any atom of the heterocycle, valence permitting, such as a carbon or nitrogen atom of the heterocycle. In some embodiments, the heterocycle is a heteroaryl. In some embodiments, the heterocycle is a heterocycloalkyl. In an exemplary embodiment, a heterocycle, e.g., pyridyl, may be fused to a saturated or unsaturated ring, e.g., cyclohexane, cyclopentane, or cyclohexene. Exemplary heterocycles include pyrrolidinyl, pyrrolyl, imidazolyl, pyrazolyl, triazolyl, piperidinyl, pyridinyl, pyrimidinyl, pyridazinyl, pyrazinyl, thiophenyl, oxazolyl, thiazolyl, morpholinyl, indazolyl, indolyl, and quinolinyl. Unless stated otherwise specifically in the specification, a heterocycle is optionally substituted by one or more substituents such as those substituents described herein.

“Heteroaryl” refers to a 3- to 12-membered aromatic ring that comprises at least one heteroatom wherein each heteroatom may be independently selected from N, O, and S. As used herein, the heteroaryl ring may be selected from monocyclic or bicyclic and fused or bridged ring systems wherein at least one of the rings in the ring system is aromatic, i.e., it contains a cyclic, delocalized (4n+2) π-electron system in accordance with the Hückel theory. The heteroatom(s) in the heteroaryl may be optionally oxidized. One or more nitrogen atoms, if present, are optionally quaternized. The heteroaryl may be attached to the rest of the molecule through any atom of the heteroaryl, valence permitting, such as a carbon or nitrogen atom of the heteroaryl. Examples of heteroaryls include, but are not limited to, azepinyl, acridinyl, benzimidazolyl, benzindolyl, 1,3-benzodioxolyl, benzofuranyl, benzooxazolyl, benzo[d]thiazolyl, benzothiadiazolyl, benzo[b][1,4]dioxepinyl, benzo[b][1,4]oxazinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, indazolyl, indolyl, indazolyl, isoindolyl, indolinyl, isoindolinyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-1H-pyrrolyl, phenazinyl, phenothiazinyl, phenoxazinyl, phthalazinyl, pteridinyl, purinyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, triazolyl, tetrazolyl, triazinyl, thieno[2,3-d]pyrimidinyl, thieno[3,2-d]pyrimidinyl, thieno[2,3-c]pridinyl, and thiophenyl (i.e. thienyl). Unless stated otherwise specifically in the specification, a heteroaryl is optionally substituted by one or more substituents such as those substituents described herein.

The term “substituted” refers to moieties having substituents replacing a hydrogen on one or more carbons or heteroatoms of the structure. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this disclosure, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, a carbocycle, a heterocycle, a cycloalkyl, a heterocycloalkyl, an aromatic and heteroaromatic moiety. In some embodiments, substituents may include any substituents described herein, for example: halogen, hydroxy, oxo (═O), thioxo (═S), cyano (—CN), nitro (—NO₂), imino (═N—H), oximo (═N—OH), hydrazino (═N—NH₂), —R^{b}—OR^{a}, —R^{b}—OC(═O)—R^{a}, —R^{b}—OC(═O)—OR^{a}, —R^{b}—OC(═O)—N(R))₂, —R^{b}—N(R^{a})₂, —R^{b}—C(═O)R^{a}, —R^{b}—C(═O)R^{a}, —R^{b}—C(═O)N(R^{a})₂, —R^{b}—O—R^{a}—C(═O)N(R^{a})₂, —R^{b}—N(R^{a})C(═O)OR^{a}, —R^{b}—N(R^{a})C(═O)R^{a}, —R^{b}—N(R^{a})S(═O)ₜR^{a} (where t is 1 or 2), —R^{b}—S(═O)ₜR^{a} (where t is 1 or 2), —R^{b}—S(═O)ₜOR^{a} (where t is 1 or 2), and —R^{b}—S(═O)ₜN(R^{a})₂ (where t is 1 or 2); and alkyl, alkenyl, alkynyl, aryl, aralkyl, aralkenyl, aralkynyl, cycloalkyl, cycloalkylalkyl, heterocycloalkyl, heterocycloalkylalkyl, heteroaryl, and heteroarylalkyl any of which may be optionally substituted by alkyl, alkenyl, alkynyl, halogen, hydroxy, haloalkyl, haloalkenyl, haloalkynyl, oxo (═O), thioxo (═S), cyano (—CN), nitro (—NO₂), imino (═N—H), oximo (═N—OH), hydrazine (═N—NH₂), —R^{b}—OR^{a}, —R^{b}—OC(═O)—R^{a}, —R^{b}—OC(═O)—OR^{a}, —R^{b}—OC(═O)—N(R))₂, —R^{b}—N(R))₂, —R^{b}—C(═O)R^{a}, —R^{b}—C(═O)OR^{a}, —R^{b}—C(═O)N(R^{a})₂, —R^{b}—O—R^{c}-C(═O)N(R^{a})₂, —R^{b}—N(R^{a})C(═O)OR^{a}, —R^{b}—N(R^{a})C(═O)R^{a}, —R^{b}—N(R^{a})S(═O)ₜR^{a} (where t is 1 or 2), —R^{b}—S(═O)ₜR^{a} (where t is 1 or 2), —R^{b}—S(═O)ₜOR^{a} (where t is 1 or 2) and —R^{b}—S(═O)ₜN(R^{a})₂ (where t is 1 or 2); wherein each R^{a} is independently selected from hydrogen, alkyl, cycloalkyl, cycloalkylalkyl, aryl, aralkyl, heterocycloalkyl, heterocycloalkylalkyl, heteroaryl, or heteroarylalkyl, wherein each R^{a}, valence permitting, may be optionally substituted with alkyl, alkenyl, alkynyl, halogen, haloalkyl, haloalkenyl, haloalkynyl, oxo (═O), thioxo (═S), cyano (—CN), nitro (—NO₂), imino (═N—H), oximo (═N—OH), hydrazine (═N—NH₂), —R^{b}—OR^{a}, —R^{b}—OC(═O)—R^{a}, —R^{b}—OC(═O)—OR^{a}, —R^{b}—OC(═O)—N(R))₂, —R^{b}—N(R))₂, —R^{b}—C(═O)R^{a}, —R^{b}—C(═O)OR^{a}, —R^{b}—C(═O)N(R^{a})₂, —R^{b}—O—R^{c}-C(═O)N(R^{a})₂, —R^{b}—N(R^{a})C(═O)OR^{a}, —R^{b}—N(R^{a})C(═O)R^{a}, —R^{b}—N(R^{a})S(═O)ₜR^{a} (where t is 1 or 2), —R^{b}—S(═O)ₜR^{a} (where t is 1 or 2), —R^{b}—S(═O)ₜOR^{a} (where t is 1 or 2) and —R^{b}—S(═O)ₜN(R^{a})₂ (where t is 1 or 2); and wherein each R^{b} is independently selected from a direct bond or a straight or branched alkylene, alkenylene, or alkynylene chain, and each RC is a straight or branched alkylene, alkenylene or alkynylene chain. In some embodiments, a substituent is selected from R²⁰ as defined herein below.

It will be understood by those skilled in the art that substituents can themselves be substituted, if appropriate. Unless specifically stated as “unsubstituted,” references to chemical moieties herein are understood to include substituted variants. For example, reference to a “heteroaryl” group or moiety implicitly includes both substituted and unsubstituted variants.

Where substituent groups are specified by their conventional chemical formulae, written from left to right, they equally encompass the chemically identical substituents that would result from writing the structure from right to left, e.g., —CH₂O— is equivalent to —OCH₂—.

“Optional” or “optionally” means that the subsequently described event of circumstances may or may not occur, and that the description includes instances where the event or circumstance occurs and instances in which it does not. For example, “optionally substituted aryl” means that the aryl group may or may not be substituted and that the description includes both substituted aryl groups and aryl groups having no substitution.

A “leaving group or atom” is any group or atom that will, under the reaction conditions, cleave from the starting material, thus promoting reaction at a specified site. Suitable examples of such groups, unless otherwise specified, include halogen atoms, mesyloxy, p-nitrobenzenesulfonyloxy and tosyloxy groups.

“Protecting group” has the meaning conventionally associated with it in organic synthesis, e.g. a group that selectively blocks one or more reactive sites in a multifunctional compound such that a chemical reaction can be carried out selectively on another unprotected reactive site and such that the group can readily be removed after the selective reaction is complete. A variety of protecting groups are disclosed, for example, in P. G. M. Wuts, Greene's Protective Groups in Organic Synthesis, Fifth Edition, 2014. For example, a hydroxy protected form is where at least one of the hydroxy groups present in a compound is protected with a hydroxy protecting group. Likewise, amines and other reactive groups may similarly be protected.

Compounds and intermediates of the present disclosure also include crystalline and amorphous forms of those compounds or intermediates, their salts or pharmaceutically acceptable salts, and active metabolites of these compounds having the same type of activity, including, for example, polymorphs, pseudopolymorphs, solvates, hydrates, unsolvated polymorphs (including anhydrates), conformational polymorphs, and amorphous forms of the compounds, as well as mixtures thereof.

Compounds or intermediates described herein may exhibit their natural isotopic abundance, or one or more of the atoms may be artificially enriched in a particular isotope having the same atomic number, but an atomic mass or mass number different from the atomic mass or mass number predominantly found in nature. All isotopic variations of the compounds of the present disclosure, whether radioactive or not, are encompassed within the scope of the present disclosure. For example, hydrogen has three naturally occurring isotopes, denoted ¹H (protium), ²H (deuterium), and ³H (tritium). Protium is the most abundant isotope of hydrogen in nature. Enriching for deuterium may afford certain therapeutic advantages, such as increased in vivo half-life and/or exposure, or may provide a compound useful for investigating in vivo routes of drug elimination and metabolism. Isotopically-enriched compounds may be prepared by conventional techniques well known to those skilled in the art.

“Isomers” are different compounds that have the same molecular formula. “Stereoisomers” are isomers that differ only in the way the atoms are arranged in space. “Enantiomers” are a pair of stereoisomers that are non-superimposable mirror images of each other. A 1:1 mixture of a pair of enantiomers is a “racemic” mixture. The term “(±)” is used to designate a racemic mixture where appropriate. “Diastereoisomers” or “diastereomers” are stereoisomers that have at least two asymmetric atoms but are not mirror images of each other. The absolute stereochemistry is specified according to the Cahn-Ingold-Prelog R-S system. When a compound is a pure enantiomer, the stereochemistry at each chiral carbon can be specified by either R or S. Resolved compounds whose absolute configuration is unknown can be designated (+) or (−) depending on the direction (dextro- or levorotatory) in which they rotate plane polarized light at the wavelength of the sodium D line. Certain compounds described herein contain one or more asymmetric centers and can thus give rise to enantiomers, diastereomers, and other stereoisomeric forms, the asymmetric centers of which can be defined, in terms of absolute stereochemistry, as (R)- or (S)-. The present chemical entities, pharmaceutical compositions and methods are meant to include all such possible stereoisomers, including racemic mixtures, optically pure forms, mixtures of diastereomers and intermediate mixtures. Optically active (R)- and (S)-isomers can be prepared using chiral synthons or chiral reagents, or resolved using conventional techniques. The optical activity of a compound can be analyzed via any suitable method, including but not limited to chiral chromatography and polarimetry, and the degree of predominance of one stereoisomer over the other isomer can be determined.

Chemical entities having carbon-carbon double bonds or carbon-nitrogen double bonds may exist in Z- or E-form (or cis- or trans-form). Furthermore, some chemical entities may exist in various tautomeric forms. Unless otherwise specified, chemical entities described herein are intended to include all Z-, E- and tautomeric forms as well.

Isolation and purification of the chemical entities and intermediates described herein can be effected, if desired, by any suitable separation or purification procedure such as, for example, filtration, extraction, crystallization, column chromatography, thin-layer chromatography or thick-layer chromatography, or a combination of these procedures. Specific illustrations of suitable separation and isolation procedures can be had by reference to the examples herein below. However, other equivalent separation or isolation procedures can also be used.

When stereochemistry is not specified, certain small molecules described herein include, but are not limited to, when possible, their isomers, such as enantiomers and diastereomers, mixtures of enantiomers, including racemates, mixtures of diastereomers, and other mixtures thereof, to the extent they can be made by one of ordinary skill in the art by routine experimentation. In those situations, the single enantiomers or diastereomers, i.e., optically active forms, can be obtained by asymmetric synthesis or by resolution of the racemates or mixtures of diastereomers. Resolution of the racemates or mixtures of diastereomers, if possible, can be accomplished, for example, by conventional methods such as crystallization in the presence of a resolving agent, or chromatography, using, for example, a chiral high-pressure liquid chromatography (HPLC) column. Furthermore, a mixture of two enantiomers enriched in one of the two can be purified to provide further optically enriched form of the major enantiomer by recrystallization and/or trituration. In addition, such certain small molecules include Z- and E-forms (or cis- and trans-forms) of certain small molecules with carbon-carbon double bonds or carbon-nitrogen double bonds. Where certain small molecules described herein exist in various tautomeric forms, the term “certain small molecule” is intended to include all tautomeric forms of the certain small molecule.

The term “salt” or “pharmaceutically acceptable salt” refers to salts derived from a variety of organic and inorganic counter ions well known in the art. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. In some embodiments, the pharmaceutically acceptable base addition salt is chosen from ammonium, potassium, sodium, calcium, and magnesium salts.

“Pharmaceutically acceptable carrier, diluent or excipient” includes without limitation any adjuvant, carrier, excipient, glidant, sweetening agent, diluent, preservative, dye, colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

The term “effective amount” or “therapeutically effective amount” refers to that amount of a compound described herein that is sufficient to affect the intended application, including but not limited to disease treatment, as defined below. The therapeutically effective amount may vary depending upon the intended treatment application (in vivo), or the subject and disease condition being treated, e.g., the weight and age of the subject, the severity of the disease condition, the manner of administration and the like, which can readily be determined by one of ordinary skill in the art. The term also applies to a dose that will induce a particular response in target cells, e.g., reduction of platelet adhesion and/or cell migration. The specific dose will vary depending on the particular compounds chosen, the dosing regimen to be followed, whether it is administered in combination with other compounds, timing of administration, the tissue to which it is administered, and the physical delivery system in which it is carried.

As used herein, “treatment” or “treating” refers to an approach for obtaining beneficial or desired results with respect to a disease, disorder, or medical condition including but not limited to a therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the subject, notwithstanding that the subject may still be afflicted with the underlying disorder. In certain embodiments, for prophylactic benefit, the compositions are administered to a subject at risk of developing a particular disease, or to a subject reporting one or more of the physiological symptoms of a disease, even though a diagnosis of this disease may not have been made.

A “therapeutic effect,” as that term is used herein, encompasses a therapeutic benefit and/or a prophylactic benefit as described above. A prophylactic effect includes delaying or eliminating the appearance of a disease or condition, delaying or eliminating the onset of symptoms of a disease or condition, slowing, halting, or reversing the progression of a disease or condition, or any combination thereof.

The term “co-administration,” “administered in combination with,” and their grammatical equivalents, as used herein, encompass administration of two or more agents to an animal, including humans, so that both agents and/or their metabolites are present in the subject at the same time. Co-administration includes simultaneous administration in separate compositions, administration at different times in separate compositions, or administration in a composition in which both agents are present.

The terms “antagonist” and “inhibitor” are used interchangeably, and they refer to a compound having the ability to inhibit a biological function (e.g., activity, expression, binding, protein-protein interaction) of a target protein or enzyme (e.g., HIF-2α). Accordingly, the terms “antagonist” and “inhibitor” are defined in the context of the biological role of the target protein. While preferred antagonists herein specifically interact with (e.g., bind to) the target, compounds that inhibit a biological activity of the target protein by interacting with other members of the signal transduction pathway of which the target protein is a member are also specifically included within this definition. A preferred biological activity inhibited by an antagonist is associated with inflammation.

The term “agonist” as used herein refers to a compound having the ability to initiate or enhance a biological function of a target protein, whether by inhibiting the activity or expression of the target protein. Accordingly, the term “agonist” is defined in the context of the biological role of the target polypeptide. While preferred agonists herein specifically interact with (e.g., bind to) the target, compounds that initiate or enhance a biological activity of the target polypeptide by interacting with other members of the signal transduction pathway of which the target polypeptide is a member are also specifically included within this definition.

“Signal transduction” is a process during which stimulatory or inhibitory signals are transmitted into and within a cell to elicit an intracellular response. A modulator of a signal transduction pathway refers to a compound which modulates the activity of one or more cellular proteins mapped to the same specific signal transduction pathway. A modulator may augment (agonist) or suppress (antagonist) the activity of a signaling molecule.

The term “heterodimerization” as used herein refers to the complex formed by the non-covalent binding of HIF-2α to HIF-1β (ARNT). Heterodimerization of HIF-2α to HIF-1β (ARNT) is required for HIF-2α DNA binding and transcriptional activity and is mediated by the HLH and PAS-B domains. Transcriptional activity following heterodimerization of HIF-2α to HIF-1β (ARNT) can affect five groups of target genes including angiogenic factors, glucose transporters and glycolytic enzymes, stem-cell factors, survival factors, and invasion factors.

The term “HIF-2α” refers to a monomeric protein that contains three conserved structured domains: basic helix-loop-helix (bHLH), and two Per-ARNT-Sim (PAS) domains designated PAS-A and PAS-B, in addition to C-terminal regulatory regions. “HIF-2α” is also alternatively known by several other names in the scientific literature, most commonly endothelial PAS domain-containing protein 1 (EPAS-1) which is encoded by the EPAS1 gene. Alternative names include basic-helix-loop-helix-PAS protein (MOP2). As a member of the bHLH/PAS family of transcription factors, “HIF-2α” forms an active heterodimeric transcription factor complex by binding to the ARNT (also known as HIF-1β) protein through non-covalent interactions.

The term “HIF-2α PAS-B domain cavity” refers to an internal cavity within the PAS-B domain of HIF-2α. The crystal structure of the PAS-B domain can contain a large (approximately 290 Å) cavity in its core. However, the amino acid side chains in the solution structure are dynamic. For example, those side chains can tend to intrude more deeply in the core, and can shrink the cavity to 1 or 2 smaller cavities or can even expand the cavity. The cavity is lined by amino acid residues comprising PHE-244, SER-246, HIS-248, MET-252, PHE-254, ALA-277, PHE-280, TYR-281, MET-289, SER-292, HIS-293, LEU-296, VAL-302, VAL-303, SER-304, TYR-307, MET-309, LEU-319, THR-321, GLN-322, GLY-323, ILE-337, CYS-339, and ASN-341 of HIF-2α PAS-B domain. The numbering system is from the known structures reported in the RCSB Protein Data Bank with PDB code 3H7W. Other numbering systems in the PDB could define the same amino acids, expressed above, that line the cavity.

The term “cell proliferation” refers to a phenomenon by which the cell number has changed as a result of division. This term also encompasses cell growth by which the cell morphology has changed (e.g., increased in size) consistent with a proliferative signal.

The term “selective inhibition” or “selectively inhibit” refers to the ability of a biologically active agent to preferentially reduce the target signaling activity as compared to off-target signaling activity, via direct or indirect interaction with the target.

“Subject” refers to an animal, such as a mammal, for example a human. The methods described herein can be useful in both human therapeutics and veterinary applications. In some embodiments, the subject is a mammal, and in some embodiments, the subject is human. “Mammal” includes humans and both domestic animals such as laboratory animals and household pets (e.g., cats, dogs, swine, cattle, sheep, goats, horses, rabbits), and non-domestic animals such as wildlife and the like.

The term “in vivo” refers to an event that takes place in a subject's body.

The term “in vitro” refers to an event that takes places outside of a subject's body. For example, an in vitro assay encompasses any assay run outside of a subject. In vitro assays encompass cell-based assays in which cells alive or dead are employed. In vitro assays also encompass a cell-free assay in which no intact cells are employed.

The following abbreviations and terms have the indicated meanings throughout:

DMSO=Dimethyl sulfoxide

### DMA=Dimethylacetamide

### DME=Dimethoxyethane

### THF=Tetrahydrofuran

### 2-MeTHf=2-Methyltetrahydrofuran

Meldrum's acid=2,2-dimethyl-1,3-dioxane-4,6-dione

### TEA=Triethylamine

### ACN=Acetonitrile

DBDMH=1,3-Dibromo-5,5-dimethylhydantoin
Dess-Martin periodinane=1,1,1-Tris(acetyloxy)-1,1-dihydro-1,2-benziodoxol-3-(1H)-one
pTsOH=p-Toluenesulfonic acid
EtOAc=Ethyl acetate
AIBN=2,2′-Azobis(2-methylpropionitrile)
oDCB=1,2-Dichlorobenzene
DBU=1,8-diazabicyclo[5.4.0]undec-7-ene
DAST=Diethylaminosulfur trifluoride

### DCM=Dichloromethane

MTBE=Methyl t-butyl ether
MEK=Methyl ethyl ketone
HATU=O-(7-azabenzotriazol-1-yl)-N,N,N′,N′-tetramethyluronium hexafluorophosphate

### NBS=N-Bromosuccinimide

NMP=N-Methyl-2-pyrrolidone
e.e. or ee=Enantiomeric excess
PPTS=Pyridinium p-toluenesulfonate

### DMAP==4-Dimethylaminopyridine

### DMF=N,N-Dimethylformamide

The term “Dess-Martin” or “Dess-Martin oxidation” refers to an oxidation reaction using Dess-Martin periodinane.

The disclosure is also meant to encompass the in vivo metabolic products of the disclosed compounds. Such products may result from, for example, the oxidation, reduction, hydrolysis, amidation, esterification, and the like of the administered compound, primarily due to enzymatic processes. Accordingly, the disclosure includes compounds produced by a process comprising administering a compound of this disclosure to a mammal for a period of time sufficient to yield a metabolic product thereof. Such products are typically identified by administering a radiolabeled compound of the disclosure in a detectable dose to an animal, such as rat, mouse, guinea pig, monkey, or to human, allowing sufficient time for metabolism to occur, and isolating its conversion products from the urine, blood or other biological samples.

### Illustrative Synthetic Schemes

Illustrative synthetic routes to prepare a compound of Formula (I), in particular the compound of Formula (I-A), shown and described herein are exemplary only and are not intended, nor are they to be construed, to limit the scope of the present disclosure in any manner whatsoever. Those skilled in the art will be able to recognize modifications of the disclosed synthetic schemes and to devise alternate routes based on the disclosed examples provided herein; all such modifications and alternate routes are within the scope of the claims.

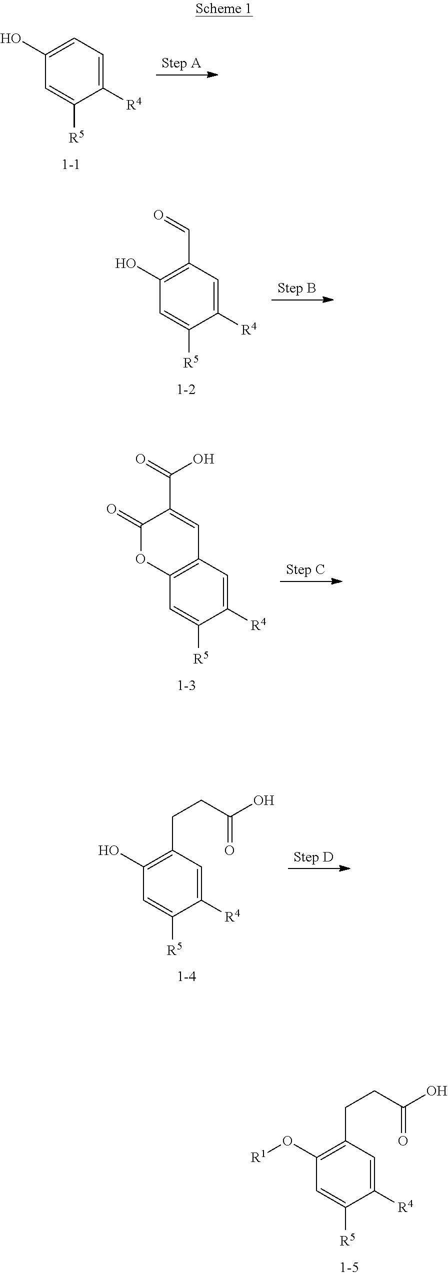

In some embodiments, a compound of Formula 1-5 can be prepared according to steps outlined in Scheme 1. Phenol 1-1 can be subjected to a formylation reaction under suitable conditions to give aldehyde 1-2. Suitable formylating reagents include paraformaldehyde. Preferably, the reaction conditions include paraformaldehyde, MgCl₂ and triethyl amine in acetonitrile at an elevated temperature, such as between 50-90° C. Aldehyde 1-2 can undergo a homologation under suitable conditions, such as Meldrum's acid and 20% K₃PO₄ in 95% EtOH:H₂O (4:1) at 15-35° C., to give carboxylic acid 1-3. Reduction and decarboxylation of acid 1-3 under suitable conditions, such as formic acid and triethylamine in DMF at 80-120° C., gives propanoic acid 1-4. Lastly, aryl ether 1-5 can be formed by coupling phenol 1-4 with R₁-LG under suitable conditions, wherein LG is a leaving group. In some examples, this coupling reaction is an S_{N}Ar reaction, conducting with R₁—F and Cs₂CO₃ in DMSO at 50-90° C. Suitable bases for the conversion of 1-4 to 1-5 include Cs₂CO₃, K₃PO₄, and K₂CO₃. Typical solvents for this reaction include DMSO and DMA. Purification of a compound of Formula 1-5 can be achieved by forming a suitable salt, including, but not limited to, an ammonium salt, a sodium salt, and a lysine salt.

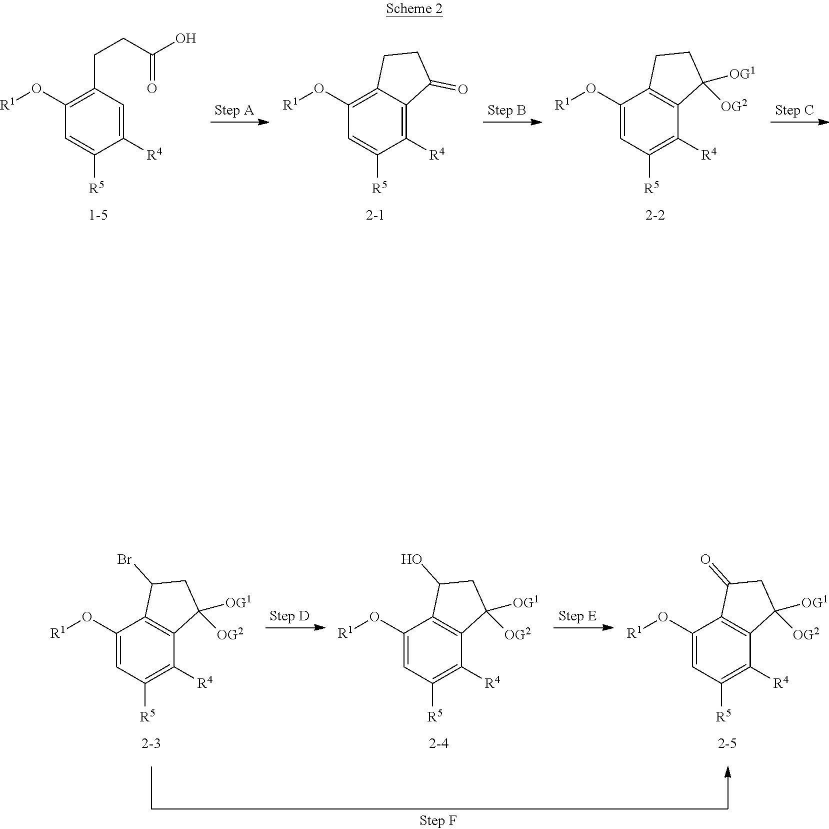

In some embodiments, a compound of Formula 2-5 can be prepared according to steps outlined in Scheme 2. Propanoic acid 1-5 can be cyclized under suitable conditions to indanone 2-1. The cyclization can proceed in two steps: formation of an acyl halide using a suitable halogenating reagent, such as (COCl)₂ or thionyl chloride in a suitable solvent (e.g., DCM), and cyclization via electrophilic aromatic substitution, such as a Friedel-Crafts acylation, in the presence of a Lewis acid (e.g., AlCl₃) at 15-35° C. Suitable solvents for the acylation include polar, aprotic solvents such as DCM or 1,2-dichlorobenzene (o-DCB). In some examples wherein R⁴ comprises a sulfane, an oxidation may be carried out to afford the corresponding sulfone. Suitable oxidizing conditions include Potassium peroxymonosulfate; peroxide and formic acid; peroxide, formic acid and sulfuric acid; TEMPO and bleach; and peroxide and sodium tungstate. The oxidation may be conducted at an elevated temperature, such as 50-70° C. In Step B, ketone 2-1 is protected to afford ketal 2-2 using a suitable alcohol or diol, such as ethylene glycol. Preferably, the reaction conditions include ethylene glycol and pTsOH, optionally with the addition of CH(OCH₃)₃ or CH(OCH₂CH₃)₃, at an elevated temperature, such as 50-70° C. Bromination of protected indanone 2-2 can proceed with a suitable brominating reagent, such as DBDMH (1,3-Dibromo-5,5-dimethylhydantoin) or NBS (N-bromosuccinimide), to give bromoindane 2-3. Formation of indanone 2-5 can proceed in either one or two steps. In the two step process, 2-3 is hydrolyzed under suitable conditions to indanol 2-4, for example, using Ag₂CO₃. Oxidation of the alcohol can afford indanone 2-5. Preferably, the oxidation is a Dess-Martin oxidation. In the one step process, bromoindane 2-3 is converted directly to indanone 2-5 under suitable conditions, such as DMSO and Et₃N or DMSO and 2,6-lutidine at an elevated temperature (e.g., 50-70° C.).

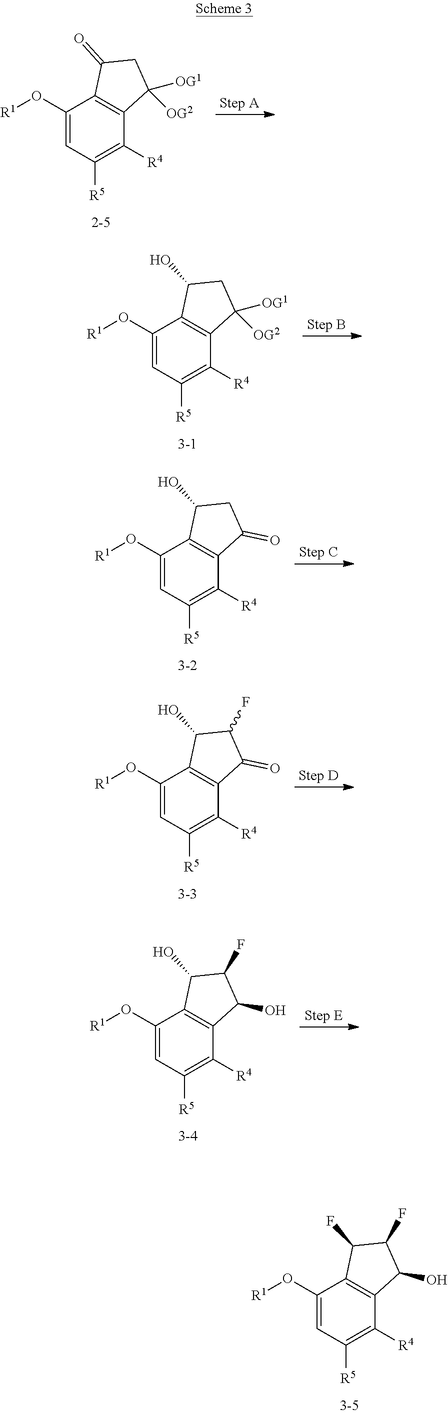

In some embodiments, a compound of Formula 3-5 can be prepared according to steps outlined in Scheme 3. Indanone 2-5 can be subjected to an asymmetric reduction, such as a Noyori reduction, under suitable conditions to give chiral indanol 3-1. Suitable catalysts include ruthenium catalysts, such as ((R,R)-2-amino-1,2-diphenylethyl)-[(4-tolyl)sulfonyl]-amido(p-cymene)-ruthenium(II) chloride. Preferably, the reaction conditions include ((R,R)-2-amino-1,2-diphenylethyl)-[(4-tolyl)sulfonyl]-amido(p-cymene)-ruthenium(II) chloride, HCO₂H and Et₃N in EtOAc at 15-35° C. Deprotection of the ketal under suitable conditions affords indanone 3-2. Suitable deprotection conditions include an acid, such as HCl, in a suitable solvent, such as acetone, MEK, or a mixture thereof. Fluorination of 3-2 with a suitable fluorinating reagent, such as Selectfluor® (1-Chloromethyl-4-fluoro-1,4-diazoniabicyclo[2.2.2]octane bis), can afford fluoroindane 3-3. Preferably, the fluorination reaction conditions include Selectfluor® and H₂SO₄ in a mixture of MeOH and MeCN (e.g., 1:1) at an elevated temperature (e.g., 50-70° C.). A second asymmetric reduction, such as a Noyori reduction, can be carried out under suitable conditions to afford trans-diol 3-4. Suitable conditions for the reduction typically include an asymmetric ruthenium catalyst, such as ((R,R)-2-amino-1,2-diphenylethyl)-[(4-tolyl)sulfonyl]-amido(p-cymene)-ruthenium(II) chloride, and a base, such as Et₃N. Preferably, the reaction conditions further comprise HCO₂H in EtOAc at 15-35° C. Lastly, fluorination of diol 3-4 under suitable conditions can give di-fluoroindanol 3-5. Suitable conditions include a fluorinating reagent, such as diethylaminosulfur trifluoride (DAST), Deoxo-Fluor® (bis(2-methoxyethyl)aminosulfur trifluoride), PyFluor, or perfluoro-1-butanesulfonyl fluoride; optionally, a base, such as DBU; and a solvent, such as THF, 2-MeTHF, DME, EtOAc, or dioxane.

### EXAMPLES

The following examples are given for the purpose of illustrating various embodiments of the invention and are not meant to limit the present disclosure in any fashion. The present examples, along with the methods described herein are presently representative of preferred embodiments, are exemplary, and are not intended as limitations on the scope of the invention. Changes therein and other uses which are encompassed within the spirit of the invention as defined by the scope of the claims will occur to those skilled in the art.

The chemical naming protocol and structure diagrams used herein are a modified form of the I.U.P.A.C. nomenclature system, using ChemDraw Professional or OpenEye Scientific Software's mol2nam application.

### Example 1: Synthesis of 3-(2-(3-cyano-5-fluorophenoxy)-5-(methylthio)phenyl)propanoic Acid

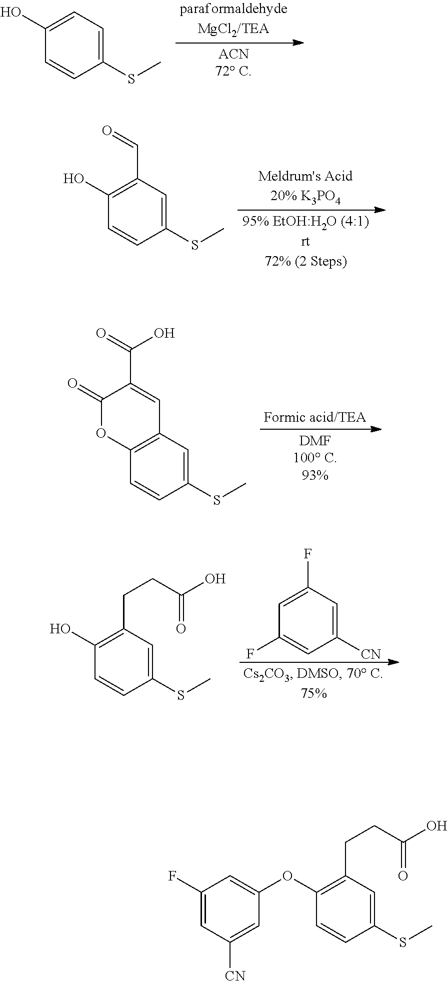

6-(Methylthio)-2-oxo-2H-chromene-3-carboxylic acid was prepared in 72% yield over two steps from 4-(methylthio)phenol via subsequent formylation and homologation reactions. Reduction and decarboxylation afforded the propanoic acid in 93% yield, which was coupled with 3,5-difluorobenzonitrile in an S_{N}Ar reaction to give the aryl ether in 75% yield.

### Example 2: Alternative synthesis of 3-(2-(3-cyano-5-fluorophenoxy)-5-(methylthio)phenyl)propanoic Acid

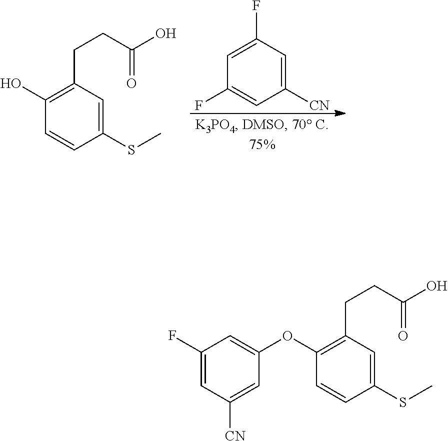 [SMILES: CSc1ccc(O)c(CCC(=O)O)c1 ; N#Cc1cc(F)cc(F)c1 ; CSc1ccc(Oc2cc(F)cc(C#N)c2)c(CCC(=O)O)c1]

3-(2-Hydroxy-5-(methylthio)phenyl)propanoic acid was prepared as described in Example 1 and coupled to 3,5-difluorobenzonitrile via an S_{N}Ar reaction in the presence of K₃PO₄ to give 3-(2-(3-cyano-5-fluorophenoxy)-5-(methylthio)phenyl)propanoic acid in 75% yield. The S_{N}Ar reaction was found to also proceed in good yield using K₂CO₃ in DMA in place of K₃PO₄ in DMSO.

### Example 3: Synthesis of 3-fluoro-5-((7-(methylsulfonyl)-3-oxo-2,3-dihydrospiro[indene-1,2′-[1,3]dioxolan]-4-yl)oxy)benzonitrile

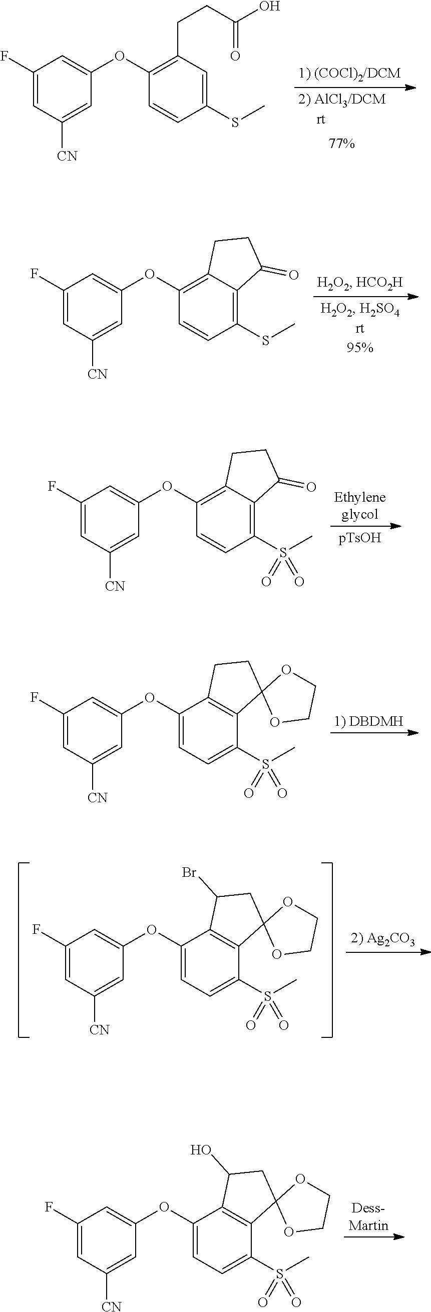

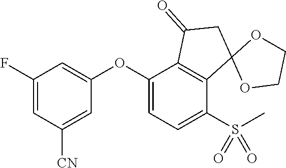 [SMILES: CS(=O)(=O)c1ccc(Oc2cc(F)cc(C#N)c2)c2c1C1(CC2=O)OCCO1]

3-(2-(3-Cyano-5-fluorophenoxy)-5-(methylthio)phenyl)propanoic acid was cyclized to the corresponding indane in 77% yield. Oxidation to the methyl sulfone was followed by protection of the ketone to give 3-fluoro-5-((7-(methylsulfonyl)-2,3-dihydrospiro[indene-1,2′-[1,3]dioxolan]-4-yl)oxy)benzonitrile. Bromination and hydrolysis gave 3-fluoro-5-((3-hydroxy-7-(methylsulfonyl)-2,3-dihydrospiro[indene-1,2′-[1,3]dioxolan]-4-yl)oxy)benzonitrile, which was oxidized to 3-fluoro-5-((7-(methylsulfonyl)-3-oxo-2,3-dihydrospiro[indene-1,2′-[1,3]dioxolan]-4-yl)oxy)benzonitrile.

### Example 4: Alternative synthesis of 3-fluoro-5-((7-(methylsulfonyl)-3-oxo-2,3-dihydrospiro[indene-1,2′-[1,3]dioxolan]-4-yl)oxy)benzonitrile

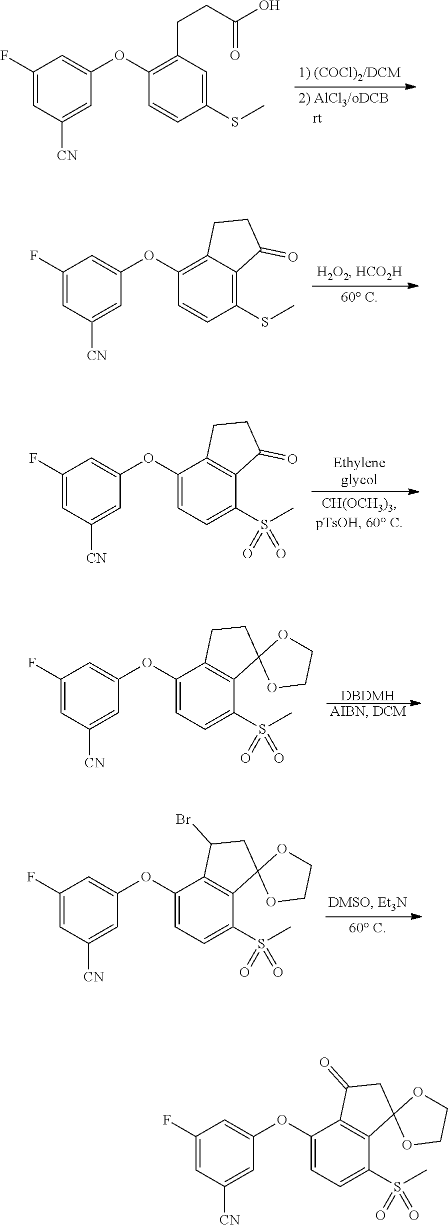

In a similar manner, 3-fluoro-5-((7-(methylsulfonyl)-3-oxo-2,3-dihydrospiro[indene-1,2′-[1,3]dioxolan]-4-yl)oxy)benzonitrile was synthesized from 3-(2-(3-cyano-5-fluorophenoxy)-5-(methylthio)phenyl)propanoic acid as outlined above. Replacing DCM from the cyclization reaction of Example 3 with 1,2-dichlorobenzene in the presence of a phase transfer catalyst (e.g., tetrabutylammonium hydrogensulfate) was found to improve the workup and impurity profile. Additionally, hydrogen peroxide and formic acid were shown to work as effectively as hydrogen peroxide, formic acid and sulfuric acid in the sulfur oxidation step. TEMPO/bleach and peroxide/sodium tungstate were also shown to be suitable alternatives to generate the desired sulfone. The addition of trimethyl orthoformate to the ketone protection step improved the purity profile. Lastly, the two step hydrolysis and oxidation procedure of Example 3 was found to proceed in a single pot with DMSO/Et₃N or DMSO/2,6-lutidine to give the title compound.

### Example 5: Synthesis of (R)-3-fluoro-5-((3-hydroxy-7-(methylsulfonyl)-1-oxo-2,3-dihydro-1H-inden-4-yl)oxy)benzonitrile

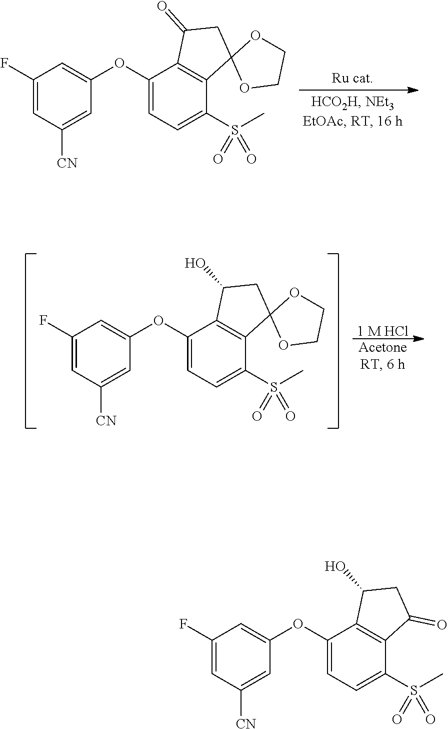

A 250 L glass-lined steel reactor was charged with ethyl acetate (159.1 kg), triethylamine (7.45 kg), formic acid (5.10 kg), 5-fluoro-3-{7′-(methylsulfonyl)-3′-oxospiro[1,3-dioxolane-2,1′-indan]-4′-yloxy}benzonitrile (14.80 kg) and ((R,R)-2-amino-1,2-diphenylethyl)-[(4-tolyl)sulfonyl]-amido(p-cymene)-ruthenium(II) chloride (0.235 kg). The mixture was stirred at 15-25° C. for 20 hours before the mixture was checked by HPLC (100.0% conversion). The reaction was extracted with 1M hydrochloric acid (74.9 kg) and 25% aqueous sodium chloride (87.6 kg). The volume was reduced to approximately 70 L by distillation under reduced pressure at 60° C. Acetone (117.0 kg) was added, and the volume was reduced to 39 L by distillation under reduced pressure at 60° C. Acetone (22.6 kg) and 1M hydrochloric acid (74.8 kg) were added, and the resulting mixture was stirred for 16 hours at 25° C. The reaction was checked by HPLC (99.7% conversion). Water (73.6 kg) was added, causing precipitation of the product. The suspension was stirred for one hour before the product was collected on a centrifuge. The product was washed with water (59.0 kg). The wet product (18.35 kg) was dried in an air vented drying cupboard at 35-40° C. for 25 hours to give a light brown solid. Yield: 11.98 kg (90.4%); ruthenium content: 632 ppm; purity 99.6%; chiral purity >99.8%.

### Example 6: Synthesis of 3-fluoro-5-(((1S,2R,3S)-2-fluoro-1,3-dihydroxy-7-(methylsulfonyl)-2,3-dihydro-1H-inden-4-yl)oxy)benzonitrile

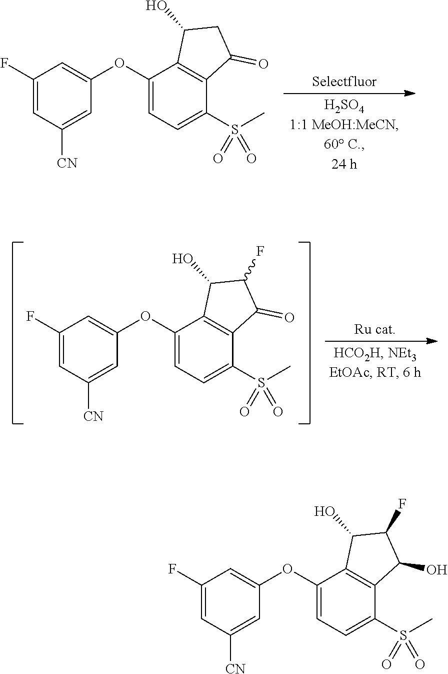

A 250 L glass-lined steel reactor was charged with acetonitrile (45.6 kg), methanol (46.2 kg) and (R)-3-fluoro-5-((3-hydroxy-7-(methylsulfonyl)-1-oxo-2,3-dihydro-1H-inden-4-yl)oxy)benzonitrile (11.65 kg) at 10-25° C. Sulfuric acid (1.65 kg) was added, and the mixture was heated to 55-60° C. Selectfluor® (5.75 kg) was added, and the reaction mixture was stirred for 1-2 h at 57-60° C. After verifying that the reaction leading to 3-fluoro-5-(((3S)-2-fluoro-3-hydroxy-7-(methylsulfonyl)-1-oxo-2,3-dihydro-1H-inden-4-yl)oxy)benzonitrile had started, another portion of Selectfluor® (11.25 kg) was added, and the reaction was continued at 57-60° C. for 17-48 h. The solvent was partly evaporated under reduced pressure. Ethyl acetate (73.4 kg) was added, and the evaporation was continued. Ethyl acetate (82.9 kg) was added, and the evaporation was continued until approximately 84 L were left in the reactor. Purified water (57.75 kg) was added at 20-50° C. The phases were separated, and the aqueous phase was back-extracted with ethyl acetate (21.2 kg). The combined organic phases were washed with 25% sodium chloride (55.6 kg).

Formic acid (2.223 kg), triethylamine (9.80 kg) and ((R,R)-2-amino-1,2-diphenylethyl)-[(4-tolyl)sulfonyl]-amido(p-cymene)-ruthenium(II) chloride (0.204 kg) were added to the organic phase, containing 3-fluoro-5-(((3 S)-2-fluoro-3-hydroxy-7-(methylsulfonyl)-1-oxo-2,3-dihydro-1H-inden-4-yl)oxy)benzonitrile. The reaction mixture was stirred for 16-24 h at 20-25° C. The reaction mixture was washed twice with 1M HCl (58.9 kg), and once with 25% sodium chloride (67.2 kg). The organic phase was filtered through a plug of silica gel (11.75 kg), and eluted with ethyl acetate (41.8 kg). The filtrate was concentrated by evaporation under reduced pressure until approximately 88 L were left in the reactor, seeded (3-fluoro-5-(((1 S,2R,3 S)-2-fluoro-1,3-dihydroxy-7-(methylsulfonyl)-2,3-dihydro-1H-inden-4-yl)oxy)benzonitrile), which did not facilitate recrystallization. The mixture was evaporated further until approximately 33 L were left in the reactor and crystallization occurred during this distillation.

n-Heptane (78.1 kg) was added, and the suspension was stirred at 38-40° C. for 30-90 min. The suspension was gradually cooled to 20-25° C. over 1-3 h, and stirred for 0-24 h, before it was isolated by centrifugation and washed with a mixture of ethyl acetate (6.5 kg) and n-heptane (11.2 kg). The wet solid was added to ethyl acetate (100.3 kg) and heated to 65-70° C. until a clear solution was obtained. Activated carbon (1.20 kg) and celite (2.15 kg) were added, and the mixture was stirred for ca. 30 minutes at 55-60° C. The suspension was filtered through a pad of celite (2.2 kg) and the filter cake was washed with ethyl acetate (21.8 kg). The filtrate was evaporated under reduced pressure until ca. 49 L of distillate was collected. n-Heptane (33.5 kg) was added at ca. 50° C., and the evaporation was resumed under reduced pressure until ca. 49 L of distillate was collected. n-Heptane (33.4 kg) was added at ca. 50° C., and the evaporation was resumed under reduced pressure until ca. 40 L of distillate was collected. The suspension was gradually cooled to 20-25° C., over 1-3 h, and stirred for 0-24 h, before it was isolated by centrifugation and washed with n-heptane (8.7 kg). The solid was dried under vacuum at 23-27° C. for at least 10 h to give a brown solid. Yield: 10.52 kg (85.6%); ruthenium content: 657 ppm; purity 96.0%.

### Example 7: Alternative synthesis of 3-fluoro-5-(((1S,2R,3S)-2-fluoro-1,3-dihydroxy-7-(methylsulfonyl)-2,3-dihydro-1H-inden-4-yl)oxy)benzonitrile

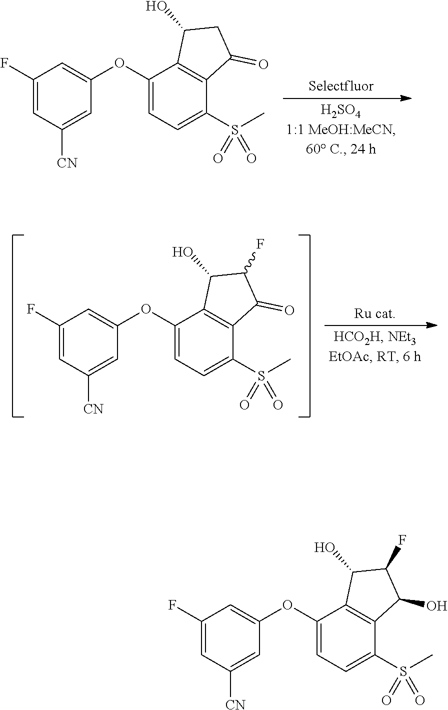

(R)-3-Fluoro-5-((3-hydroxy-7-(methylsulfonyl)-1-oxo-2,3-dihydro-1H-inden-4-yl)oxy)benzonitrile (10.50 kg, 29.1 mop was added to a mixture of acetonitrile (41.3 kg) and methanol (41.5 kg) at 10-25° C. Sulfuric acid (1.49 kg) was added, and the mixture was heated to 55-60° C. Selectfluor® (5.15 kg, 14.5 mol) was added, and the reaction mixture was stirred for 1-2 h at 57-60° C. After verifying that the reaction to give 3-fluoro-5-(((3S)-2-fluoro-3-hydroxy-7-(methylsulfonyl)-1-oxo-2,3-dihydro-1H-inden-4-yl)oxy)benzonitrile had started, another portion of Selectfluor® (10.29 kg, 29.0 mol) was added, and the reaction was continued at 57-60° C. for 17-24 h. The solvent was partly evaporated under reduced pressure. Ethyl acetate was added, and the evaporation was continued. Ethyl acetate was added, and the evaporation was continued until approximately 76 L was left in the reactor. Purified water was added at 20-50° C. The phases were separated, and the aqueous phase was back-extracted with ethyl acetate. The combined organic phases were washed with 25% sodium chloride.

Formic acid (2.003 kg, 43.5 mol), triethylamine (8.80 kg, 87.0 mol) and ((R,R)-2-amino-1,2-diphenylethyl)-[(4-tolyl)sulfonyl]-amido(p-cymene)-ruthenium(II) chloride (0.092 kg, 0.15 mol) were added to the organic phase (containing 3-fluoro-5-(((3S)-2-fluoro-3-hydroxy-7-(methylsulfonyl)-1-oxo-2,3-dihydro-1H-inden-4-yl)oxy)benzonitrile). The reaction mixture was stirred for 16-24 h at 20-25° C. The reaction mixture was washed twice with 1M HCl, and once with 25% sodium chloride. The organic phase was diluted with ethyl acetate (26.6 kg) and activated carbon (4.9 kg) was added and the mixture was stirred for 22-24 hours at 58-63° C. The suspension was filtered through a pad of celite (5.3 kg) and the filter cake was washed with ethyl acetate (40.4 kg). The filtrate was evaporated under reduced pressure until ca. 28 L of residue was left. n-Heptane (43.6 kg) was added at 58-63° C. The suspension was gradually cooled to 20-25° C. over 1-3 h, and stirred for 0-24 h, before it was isolated by centrifugation and washed with a mixture of ethyl acetate (6.5 kg) and n-heptane (10.0 kg). The solid was dried under vacuum at 38-42° C. for at least 10 h, to give 9.6 kg of 3-fluoro-5-(((1S,2R,3S)-2-fluoro-1,3-dihydroxy-7-(methylsulfonyl)-2,3-dihydro-1H-inden-4-yl)oxy)benzonitrile (87% yield) as a brown solid.

### Example 8: Synthesis of 3-(((1S,2S,3R)-2,3-difluoro-1-hydroxy-7-(methylsulfonyl)-2,3-dihydro-1H-inden-4-yl)oxy)-5-fluorobenzonitrile

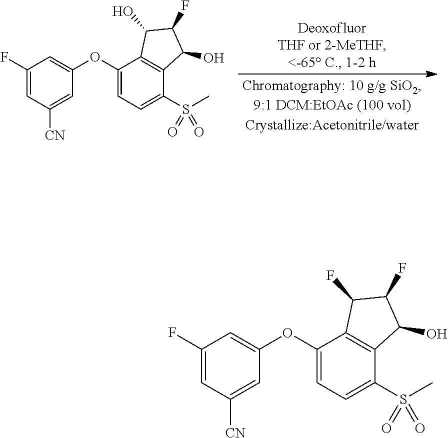 [SMILES: CS(=O)(=O)c1ccc(Oc2cc(F)cc(C#N)c2)c2c1[C@H](O)[C@H](F)[C@H]2O ; CS(=O)(=O)c1ccc(Oc2cc(F)cc(C#N)c2)c2c1[C@H](O)[C@H](F)[C@@H]2F]

A 60 L stainless steel reactor was charged with 2-methyltetrahydrofuran (69.0 kg) and 3-fluoro-5-(((1 S,2R,3 S)-2-fluoro-1,3-dihydroxy-7-(methylsulfonyl)-2,3-dihydro-1H-inden-4-yl)oxy)benzonitrile (10.00 kg) at 15-25° C. The solution was cooled to −69 to −75° C., and 50% Deoxo-Fluor® in toluene (15.25 kg) was added, while the temperature was maintained at −69 to −75° C. The reaction mixture was stirred for 60-120 minutes at −69 to −75° C. In-process control verified >85% conversion of 3-fluoro-5-(((1S,2R,3S)-2-fluoro-1,3-dihydroxy-7-(methylsulfonyl)-2,3-dihydro-1H-inden-4-yl)oxy)benzonitrile into 3-(((1S,2S,3R)-2,3-difluoro-1-hydroxy-7-(methylsulfonyl)-2,3-dihydro-1H-inden-4-yl)oxy)-5-fluorobenzonitrile and a minimum 60% (AUC) of 3-(((1S,2S,3R)-2,3-difluoro-1-hydroxy-7-(methylsulfonyl)-2,3-dihydro-1H-inden-4-yl)oxy)-5-fluorobenzonitrile in the HPLC analysis. Methanol (0.798 kg) was added, and the mixture was stirred for 10-30 minutes before the mixture was quenched into 6% sodium hydrogen carbonate (124.2 kg). To the resulting mixture was added ethyl acetate (90.2 kg), and the phases were separated at 23-27° C. The organic phase was washed with 6% sodium hydrogen carbonate (124.05 kg) and 0.5 M hydrochloric acid (103.9 kg). The solvent was partly evaporated under reduced pressure until approximately 72 L were left in the reactor. Silica gel 60 (40-63 μm, 15.35 kg) and toluene (63.3 kg) were added, and the evaporation was continued under reduced pressure until approximately 72 L were left in the reactor. Toluene (40.2 kg) was added, and the evaporation was continued under reduced pressure until approximately 72 L were left in the reactor. The silica gel suspension containing crude 3-(((1S,2S,3R)-2,3-difluoro-1-hydroxy-7-(methylsulfonyl)-2,3-dihydro-1H-inden-4-yl)oxy)-5-fluorobenzonitrile was loaded to a silica gel column prepared from silica gel 60 (40-63 μm, 67.4 kg), toluene (145.2 kg) and methanol (1.349 kg). The reactor was washed with first 2% (v/v) methanol in toluene (8.97 kg) followed by 2% (v/v) methanol in dichloromethane (27.127 kg)—the washes were transferred to the column. The column was eluted with 2% (v/v) methanol in dichloromethane (1328.2 kg). The top part of the column was broken up/resuspended in eluent, five times in order to dissolve all the precipitated product. The collected fractions were analyzed by TLC, and fractions containing 3-(((1S,2S,3R)-2,3-difluoro-1-hydroxy-7-(methylsulfonyl)-2,3-dihydro-1H-inden-4-yl)oxy)-5-fluorobenzonitrile were then analyzed by HPLC. Sufficiently pure fractions were combined, and the solvent was partly evaporated under reduced pressure until approximately 50 L were left in the reactor. Acetonitrile (108.3 kg) was added, and the evaporation was continued under reduced pressure until approximately 64 L were left in the reactor. Purified water (64.20 kg) was added at 50-55° C. The suspension was stirred at 50-55° C. for at least 10 hours and then checked with HPLC for any residual impurity at RRT 1.85.

The suspension was cooled to 20-25° C. and stirred for 2-4 hours before the crude 3-(((1 S,2 S,3R)-2,3-difluoro-1-hydroxy-7-(methylsulfonyl)-2,3-dihydro-1H-inden-4-yl)oxy)-5-fluorobenzonitrile was isolated by centrifugation and washed with purified water/acetonitrile (1:1 (v/v), 18.85 kg).

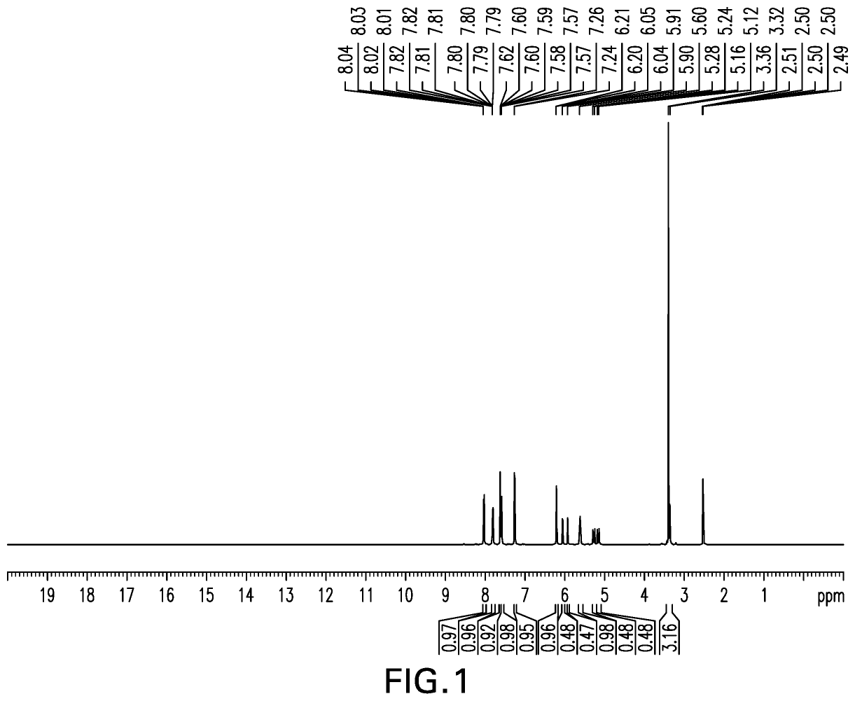

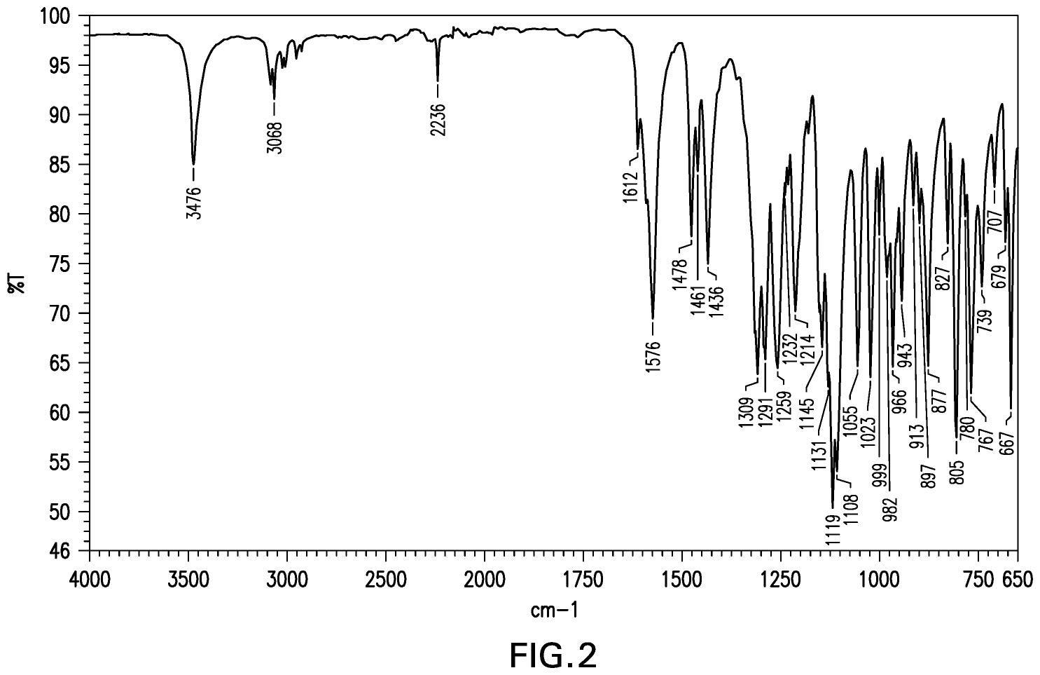

A 100 L glass reactor was charged with crude 3-(((1S,2S,3R)-2,3-difluoro-1-hydroxy-7-(methylsulfonyl)-2,3-dihydro-1H-inden-4-yl)oxy)-5-fluorobenzonitrile (wet 6.0 kg corresponding to 5.35 kg of dry material) and acetonitrile (31.2 kg). The mixture was heated to 67° C. until a clear solution was obtained, and a polish filtration was applied. The filter was washed with acetonitrile (2.35 kg). Purified water (43.1 kg) was added at 65-70° C. to the filtered solution. The mixture was stirred for 4-5 hours at 65-70° C. and was then cooled to 20-25° C. The mixture was stirred for 13 hours at 20-25° C. before 3-(((1S,2S,3R)-2,3-difluoro-1-hydroxy-7-(methylsulfonyl)-2,3-dihydro-1H-inden-4-yl)oxy)-5-fluorobenzonitrile was isolated by centrifugation and washed with purified water (15.2 kg). The wet solid was analyzed by HPLC and no new impurities were found. The solid was dried under vacuum at 45-50° C. for 20 hours to give a light yellowish solid, the identity of which was confirmed by FTIR and HPLC by comparison to a reference standard (m/z=406.0344 [M+Na]⁺; m/z=789.0790 [2M+Na]⁺). Yield: 4.783 kg (47.6%); purity >99.9%; chiral purity >99.8% ee. ¹H NMR and FTIR spectra of the reference standard are provided in FIG. 1 and FIG. 2, respectively.

### Example 9: Alternative synthesis of 3-(((1S,2S,3R)-2,3-difluoro-1-hydroxy-7-(methylsulfonyl)-2,3-dihydro-1H-inden-4-yl)oxy)-5-fluorobenzonitrile

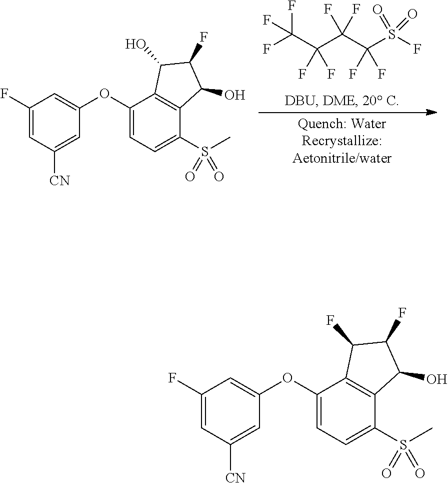 [SMILES: CS(=O)(=O)c1ccc(Oc2cc(F)cc(C#N)c2)c2c1[C@H](O)[C@H](F)[C@H]2O ; O=S(=O)(F)C(F)(F)C(F)(F)C(F)(F)C(F)(F)F ; CS(=O)(=O)c1ccc(Oc2cc(F)cc(C#N)c2)c2c1[C@H](O)[C@H](F)[C@@H]2F]

A 250 L glass-lined steel reactor was charged with 1,2-dimethoxyethane (42.4 kg) and 3-fluoro-5-(((1 S,2R,3 S)-2-fluoro-1,3-dihydroxy-7-(methylsulfonyl)-2,3-dihydro-1H-inden-4-yl)oxy)benzonitrile (6.10 kg) at 15-20° C. Perfluoro-1-butanesulfonyl fluoride (5.50 kg) was added in one portion. A solution of 1,8-diazabicyclo[5.4.0]undec-7-ene (2.90 kg) in 1,2-dimethoxyethane (10.60 kg) was added over 35 minutes at 15-20° C. The mixture was stirred at 15-20° C. for 30 minutes and then checked by HPLC. Purified water (61.25 kg) was added and the suspension was stirred for 20 hours at 15-20° C., before the crude product was isolated by centrifugation and washed with a mixture of 1,2-dimethoxyethane (5.30 kg) and purified water (6.15 kg).

The wet solid (6.35 kg) was dissolved in acetonitrile (33.6 kg). The mixture was heated to 71.9° C. and polish filtered into a clean reactor. The polish filter was washed with acetonitrile (9.6 kg) and the wash was transferred to the reactor with the initial filtrate. The solution was concentrated at 60-80° C., down to a volume of 25 L and then added polish filtered purified water (36.00 kg). The suspension was cooled to 15-20° C. and stirred at this temperature for 2 hours. The product was collected by centrifugation and washed with polish filtered purified water (12.30 kg). The product was dried in an air vented dryer at 40-50° C. for 22 hours to give a white to brown solid, the identity of which was confirmed by FTIR and HPLC by comparison to a reference standard (m/z=406.0344 [M+Na]⁺; m/z=789.0790 [2M+Na]⁺). Yield: 4.074 kg (66.5%). ¹H NMR and FTIR spectra of the reference standard are provided in FIG. 1 and FIG. 2, respectively.

### Example 10: Alternative synthesis of 3-(((1S,2S, 3R)-2,3-difluoro-1-hydroxy-7-(methylsulfonyl)-2,3-dihydro-1H-inden-4-yl)oxy)-5-fluorobenzonitrile

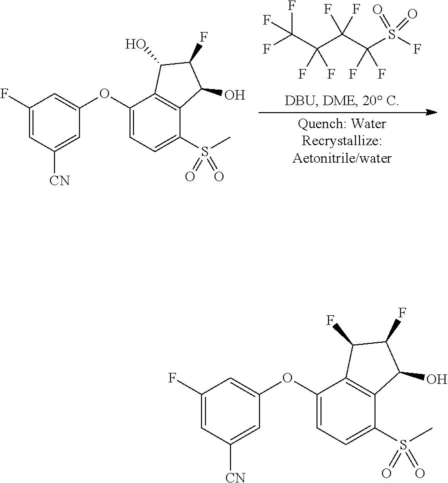 [SMILES: CS(=O)(=O)c1ccc(Oc2cc(F)cc(C#N)c2)c2c1[C@H](O)[C@H](F)[C@H]2O ; O=S(=O)(F)C(F)(F)C(F)(F)C(F)(F)C(F)(F)F ; CS(=O)(=O)c1ccc(Oc2cc(F)cc(C#N)c2)c2c1[C@H](O)[C@H](F)[C@@H]2F]

A 250 L glass-lined reactor was charged with 1,2-dimethoxyethane (62.5 kg) and 3-fluoro-5-(((1 S,2R,3 S)-2-fluoro-1,3-dihydroxy-7-(methylsulfonyl)-2,3-dihydro-1H-inden-4-yl)oxy)benzonitrile (9.0 kg; 23.6 mol) at 15-20° C. The mixture was cooled to 2-8° C., before perfluoro-1-butanesulfonyl fluoride (9.3 kg; 30.8 mol; 1.30 eq.) was added in one portion. A. solution of 1,8 diazabicyclo[5.4.0]undec-7-ene (3.6 kg; 23.7 mol; 1.0 eq.) in 1,2-dimethoxyethane (15.6 kg) was added aver 30-45 minutes at 2-8° C. The mixture was stirred at 2-8° C. for 20-45 minutes and checked by HPLC. A solution of 1,8 diazabicyclo[5.4.0]undec-7-ene (1.08 kg) in 1,2-dimethoxyethane (1.1 kg) was added over 5-15 minutes at 2-8° C. The mixture was stirred at 2-8° C. for 20-45 minutes and checked by HPLC. Water (0.9 kg) was added and the reaction mixture was heated to 20° C., before water (73.8 kg) was added over 30-45 minutes, 1,8 Diazabicyclo[5.4.0]undec-7-ene (0.17 kg) was added. The suspension was heated to 60° C. and stirred at this temperature for 30-45 minutes before the suspension was cooled to 20-25° C. over 2 hours. The mixture was stirred aver night at 20-25° C., before the product was collected by centrifugation and washed with DME/water (55:45 v/v; 16.7 kg) followed by water (9.0 kg).

The wet solid was suspended in acetonitrile (41.5 kg) and heated to 60° C. to give a clear solution. The solution was polish filtered and the polish filter was washed with acetonitrile (20.7 kg). The wash and filtrate were combined and concentrated under reduced pressure using a jacket temperature of ca. 60° C. (50 L of distillate was collected). The slightly turbid reaction mixture was heated to 60° C. and then water (29.3 kg) was added over 30-45 minutes. The resulting suspension was stirred for 30-45 minutes at 60° C. and then slowly cooled to 20-25° C. over 3 hours. The mixture was stirred over night at 20-25° C., before the product was collected by centrifugation and washed with water (13.0 kg). The crystallization described above was repeated if necessary based on HPLC analysis. The product was dried in a vacuum dryer at 38-42° C. for at least 10 hours to give 3-(((1S,2S,3R)-2,3-difluoro-1-hydroxy-7-(methylsulfonyl)-2,3-dihydro-1H-inden-4-yl)oxy)-5-fluorobenzonitrile (5.8 kg, 64% yield).

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1-2. (canceled)

3-15. (canceled)

16-18. (canceled)

19-34. (canceled)

35-36. (canceled)

37-60. (canceled)

61-66. (canceled)

67-71. (canceled)

72. (canceled)

73. A compound of Formula (IV-A):

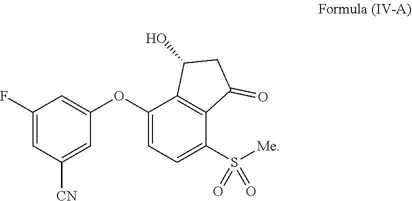 [SMILES: CS(=O)(=O)c1ccc(Oc2cc(F)cc(C#N)c2)c2c1C(=O)C[C@H]2O]

74. (canceled)

75. A compound of Formula (III-A):

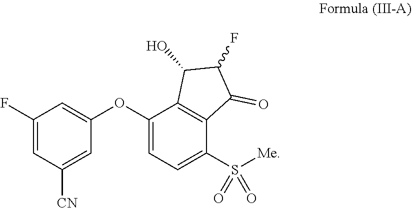 [SMILES: CS(=O)(=O)c1ccc(Oc2cc(F)cc(C#N)c2)c2c1C(=O)C(F)[C@H]2O]

76-80. (canceled)
